(19) United States
(12) Patent Application Publication
(10) Pub. No.: US 2024/0302368 A1
(43) Pub. Date: Sep. 12, 2024

(54) MIGRATION OF HUMAN PERIPHERAL LEUKOCYTES IN RESPONSE TO CHEMOTACTIC FACTORS DURING PREGNACY

(71) Applicant: The Governors of the University of Alberta, Edmonton (CA)

(72) Inventors: David Mark OLSON, Edmonton (CA); Kelycia Brynn LEIMERT, Edmonton (CA); Jennifer Anne ARENS-GUBBELS, Canton, SD (US); Han Hyung LEE, Edmonton (CA); Xin FANG, Malahat (CA); Tania Gisselle Rodezno ANTUNES, Edmonton (CA); Jun TAKEDA, Bunkyo-Ku, Tokyo (JP)

(21) Appl. No.: 18/574,221

(22) PCT Filed: Jun. 3, 2022
(86) PCT No.: PCT/CA2022/050888

§ 371 Date: Dec. 26, 2023

Related U.S. Application Data
(60) Provisional application No. 63/216,682, filed on Jun. 30, 2021.

(51) Int. Cl. G01N 33/569; A61K 38/17; A61P 15/06

(52) U.S. Cl. CPC G01N 33/56972; A61K 38/1793; A61P 15/06

(57) ABSTRACT

Migration of human peripheral leukocytes in response to chemotactic factors during pregnancy. The methods include determining the likelihood of a pregnant subject undergoing delivery within about 7 days by obtaining peripheral leukocytes from the pregnant subject and determining a chemotactic activity of the leukocytes, wherein an increased likelihood of delivery within about 7 days is indicated when the chemotactic activity is at a level characteristic of a full term pregnancy. Chemotactic activity is assessed by measuring migration of peripheral leukocytes towards a chemoattractant which is a factor present in a sample comprising human fetal membrane obtained from a full term pregnant subject. Other biomarkers or F-actin polarization of peripheral leukocytes are also indicative of the likelihood of a pregnant subject undergoing delivery. Further methods disclosed include minimizing complications of preterm delivery by administration of an IL-1 antagonist when there is a likelihood of preterm delivery.

### SEQUENCE LISTING

The instant application contains a Sequence Listing which has been previously submitted electronically in .TXT file format in the priority International Application No. PCT/CA2022/050888 and is hereby incorporated by reference in its entirety. Said .TXT copy, created Jun. 15, 2022, is named pctca2023050888-seql and is 1,983 bytes in size.

### FIELD

The present disclosure relates generally to migration of human peripheral leukocytes in response to chemotactic factor during pregnancy.

### BACKGROUND

Leukocytes invade the human uterus throughout pregnancy for various purposes to facilitate implantation, pregnancy maintenance and termination, uterine involution and postpartum cervical remodeling. A specific body of research evidence suggests that peripheral circulating leukocytes invade human and animal intrauterine tissues at term and preterm birth (PTB) to effect labor. In the human myometrium and decidua, neutrophils, macrophages, and T-lymphocytes invade during term labour (TL), and in the cervix, leukocytes infiltrate the tissue at term. In mice, both full- and sub-PTB-inducing doses of the gram-negative bacterial mimic, lipopolysaccharide (LPS), were sufficient to stimulate neutrophil infiltration of fetal membranes. This invasion was blocked with rytvela, an interleukin (IL)-1 receptor antagonist indicating a critical, upstream role for IL-1 β. Once these leukocytes invade, it has been proposed that they release an array of matrix metalloproteases, prostaglandins, cytokines, chemokines and other effectors that not only amplify this inflammatory event, but also facilitate the uterine transition from pregnancy to labor through activation of the uterus for labor (e.g. altered expression of uterine activation proteins, remodeling of the cervical extracellular matrix, and breakdown of the fetal membranes), which culminate in parturition.

Predicting the timing of labour onset or delivery is an international unmet medical need.

### SUMMARY

In one aspect, there is provided a method of determining the likelihood of a pregnant subject undergoing delivery within about 7 days, comprising:
obtaining peripheral leukocytes from said pregnant subject; and
determining a chemotactic activity of said leukocytes, wherein an increased likelihood of delivery within about 7 days is indicated when said chemotactic activity of said leukocytes is at a level characteristic of a full term pregnancy, wherein determining said chemotactic activity comprises measuring migration of said leukocytes towards a chemoattractant, wherein said chemoattractant is a factor present in a sample comprising hFM obtained from a full term pregnant subject.

In one example, wherein the pregnant subject is at least 35 weeks pregnant.

In one example, wherein the pregnant subject is less than 35 weeks pregnant.

In one example, wherein the pregnant subject is at risk of preterm delivery.

In one example, wherein the pregnant subject has ruptured membranes.

In one example, wherein said leukocyte is a granulocyte, T-lymphocyte, monocyte, macrophage, NK cell, or B-lymphocyte.

In one example, wherein said leukocyte is a neutrophil.

In one example, said sample further comprising IL1β.

In one example, wherein said subject is a human.

In one aspect, there is provided a method of treating a pregnant subject to minimize complications of a preterm delivery, comprising:
obtaining peripheral leukocytes from said pregnant subject; and
determining a chemotactic activity of said leukocytes, wherein an increased likelihood of delivery within about 7 days is indicated when said chemotactic activity of said leukocytes is at a level characteristic of a full term pregnancy, wherein determining said chemotactic activity comprises measuring migration of said leukocytes towards a chemoattractant, wherein said chemoattractant is a factor present in a sample comprising hFM obtained from a full term pregnant subject; and
administering an IL-1 receptor (IL-1R) antagonist to said pregnant when there is a likelihood of preterm delivery.

In one example, wherein said IL-1R antagonist is rytvela.

In one example, wherein there is a likelihood of delivery before about 35 weeks of gestation.

In one example, wherein the pregnant subject is less than 35 weeks pregnant.

In one example, wherein the pregnant subject has ruptured membranes.

In one example, wherein said leukocyte is a granulocyte, T-lymphocyte, monocyte, macrophage, NK cell, or B-lymphocyte.

In one example, wherein said leukocyte is a neutrophil.

In one example, said sample further comprising IL1β.

In one example, wherein said subject is a human.

In one aspect, there is provided a method of determining the likelihood of a pregnant subject undergoing delivery within about 7 days, comprising:
obtaining peripheral leukocytes from said pregnant subject; and
obtaining a sample from a pregnant subject; contacting the sample with a reagent to a biomarker, to form a complex between the agent and the biomarker present in the sample; measuring the complex formed to determine the amount or concentration of said biomarker in the sample; wherein an increased likelihood of delivery within about 7 days is indicated when said chemotactic activity of said leukocytes is at a level characteristic of a full term pregnancy, wherein determining said chemotactic activity comprises measuring migration of said leukocytes towards a chemoattractant, wherein said chemoattractant is a factor present in a sample comprising hFM obtained from a full term pregnant subject.

In one example, wherein the biomarker is CXCR2, CXCR3, CXCR5, CCR1, CCR3, CCR5, CX3CR1 and/or CCR7.

In one example, wherein the biomarker is PI3KCB, PI3KCD, Rac1, Vav1, Arp2, and/or Arp3.

In one example, wherein the sample is a serum sample.

In one example, wherein the pregnant subject is at least 35 weeks pregnant.

In one example, wherein the pregnant subject is less than 35 weeks pregnant.

In one example, wherein the pregnant subject is at risk of preterm delivery.

In one example, wherein the pregnant subject has ruptured membranes.

In one example, wherein said subject is a human.

In one aspect, there is provided a method of determining the likelihood of a pregnant subject undergoing delivery within about 7 days, comprising:
obtaining peripheral leukocytes from said pregnant subject; and
determining a F-actin polarization of said leukocytes, wherein an increased likelihood of delivery within about 7 days is indicated when said F-actin polarization of said leukocytes is at a level characteristic of a full term pregnancy.

In one example, wherein the pregnant subject is at least 35 weeks pregnant.

In one example, wherein the pregnant subject is less than 35 weeks pregnant.

In one example, wherein the pregnant subject is a risk of preterm delivery.

In one example, wherein the pregnant subject has ruptured membranes.

In one example, wherein said leukocyte is a granulocyte, T-lymphocyte, monocyte, macrophage, NK cell, or B-lymphocyte.

In one example, wherein said leukocyte is a neutrophil.

In one example, wherein said subject is a human.

### BRIEF DESCRIPTION OF THE FIGURES

Embodiments of the present disclosure will now be described, by way of example only, with reference to the attached Figures.

FIG. 1. Longitudinal leukocyte migration throughout pregnancy (A, B) or categorical (event) groupings (C-E) in response to term labor (TL, A-D) and preterm labor (PL, E) chemoattractant. In longitudinal studies from women throughout pregnancy, the migration was slightly higher during the second trimester (red line is computed mean value) (A). From 35 to 40 wk leukocyte migration rose significantly (r2=0.9952, p<0.01). (B). Mean trimester migration values were highest in the second trimester and lowest at 3 days postpartum (C). When stratified by preterm not in labor (PNL), PL, term not in labor (TNL), or TL, the highest attraction was with PNL and the lowest with PL leukocytes (D). PL chemoattractant demonstrates greater migration response from PL leukocytes than TL leukocytes (E). *p<0.05, **p<0.01, *** p<0.001, n=3-20 for each bar.

FIG. 2. Comparison of 40 cytokines/chemokines from TNL and TL maternal serum. Serum was isolated from whole blood using centrifugation. Serum components were quantified using multiplex screening analysis. *p<0.05, ***p<0.001 (n=3 for each bar). Statistically significant differences are indicated by arrows.

FIG. 3. Relative mRNA abundance for several chemokine receptors (X axis) correlates with the migration of term leukocytes to term chemoattractant (Y axis) in TL leukocytes. Correlation between the number of leukocytes migrated and the relative mRNA abundance for each gene was explored by Pearson's Correlation Test (n=12).

FIG. 4. mRNA expression of chemokine receptors on peripheral human leukocytes at PNL, PL, TNL and TL. Several of the chemokines demonstrate significant increases from PNL to TNL and to TL. *p<0.05, **p<0.01, n=6 for each bar.

FIG. 5. Relative mRNA abundance for six intracellular signaling molecules correlate with the chemotaxis of TL leukocytes to TL chemoattractant. Correlation between the number of leukocytes migrated and the relative mRNA abundance for each gene was explored by Pearson's Correlation Test (n=14).

FIG. 6. Polarization of F-actin in TL neutrophils but not in PNL or TNL. Top: F-actin was stained with Rhodamine phalloidin (red) and nuclei were stained with DAPI (blue). Cells were viewed using an Olympus Fluoview FV1200 confocal microscope. Fifty or more cells on each of 3-5 sides from each participant were visualized and assessed. Trained observers (at least 2 per slide) were blinded as to pregnancy group, and slides were randomized. Cells were stratified into focal or non-focal groups. Bottom: n=6-8 participants per group. Different superscripts signify statistical significance, p<0.03, ANOVA with Tukey's test.

FIG. 7. Conditioned medium from hFM explants and TL maternal serum activate leukocytes for migration. Leukocytes obtained from TNL women were exposed to conditioned medium from TNL and TL explants −/+ IL-1β. Both TNL (NS) and TL (p<0.01) explant medium exposure for 1 h increased leukocyte migration in response to TL chemoattractant; IL-1 β exposure had no effect (A). Leukocytes obtained from TNL women were exposed to serum from TNL and TL women for 1 h and then tested for migration to either TNL or TL chemoattractant. Both TNL and TL serum incubation significantly increased TNL leukocyte migration in response to TL chemoattractant (B). *p<0.05, **p<0.01, ***p<0.001 (n=6 each bar).

FIG. 8. hFM explants and peripheral leukocytes secrete chemotactic factors. Conditioned medium from TNL and TL hFM explants treated −/+ IL-1 β was used to attract TL leukocytes in LMA; IL-1β had no effect on enhancing chemoattractant from hFM explants (A). Conditioned medium from TL leukocytes was prepared by co-incubation −/+ IL-1 β plus TNL or TL hFM explant conditioned medium. This double-conditioned medium was tested as a chemoattractant for TL leukocyte migration. TL leukocyte chemoattractant significantly increased TL leukocyte migration and IL-1 β co-treatment further increased migration (B). *p<0.05, **p<0.01, ***p<0.001 (n=6 for each bar).

FIG. 9. Mostly neutrophils migrate in the Boyden chamber. Flow cytometry using antibodies (CD11b, CD14, CD3, and CD4) to assess the relative proportions of cells found in the isolated leukocytes from term labor women placed into the upper Boyden chamber were identified by setting specific gates for each leukocyte population based on dot-plot graphs of side-scatter versus forward scatter (A). Approximately 99% of migrated leukocytes were neutrophils or other cells detecting positive to CD11b+ (B).

FIG. 10. Performance of the leukocyte migration assay using plastic Boyden chambers printed on a 3D printer (A). The migration patterns of the assay produced distinct cut-off values for participants who would deliver within seven days at 4575 total migrated cells. The receiver-operator characteristics curve is shown with an area under the curve (AUC) of 83% (B). The cost-curve plot of the migration data demonstrates that the probability cost function (X axis) produces the highest normalized expected cost or error rate of 20% (Y axis) when the probability cost is low, around 30%, and the lowest error rate (10%) when the probability cost is highest at 80%. These parameters define the operating range of the probability cost since the curve diverts from the distribution of the yellow and black-dotted line that describes trivial classifiers. The straight line nature of the curve between 30% and 80% indicates that a single threshold value, or one LMA assessment, yields these levels of performance (C; courtesy of Dr. R Holte). There is no change in the quality of performance of the assay using printed 3D chambers (D, Chongqing) when compared to previously published results that used commercial Boyden chambers (D, Edmonton) (19). PPV: positive predictive value; NPV: negative predictive value.

FIG. 11. Effect of IL-1β and rytvela on preterm delivery and neutrophil invasion of mouse uterus and fetal brain (A) PTB occurs only in IL-1 β-stimulated mice. This is inhibited by co-administration of rytvela (n=5-7 per group). (B) Representative images of GD17 fetuses. Those dams treated 24 h earlier with IL-1β alone (centre) were undergoing resorption whereas dams co-treated with rytvela had normal GD 17 fetuses. (C) Counts of neutrophils per high powered field (20× optical zoom) in mouse lower uterus (n=5-7). (D) Representative histological images of the mouse lower uterus stained for DNA (Hoechst 33342, red stain) and a neutrophil surface marker (anti-Ly6g, green stain). (E) Counts of neutrophils per high powered field (20× optical zoom) in mouse fetal brains (n=3 dams per group, n=2 fetuses per dam). (F) Representative histological images of the mouse fetal brain stained for DNA (Hoechst 33342, red stain) and a neutrophil surface marker (anti-Ly6g, green stain). *p<0.05, *p<0.01.

FIG. 12. Intrauterine tissue chemotactic factor in mice during late gestation using human TL leukocytes in LMA. (A) Total protein was extracted from mouse intrauterine tissues at gestational day (GD)15, 18.5 or 19 and pooled (n=6 per GD). Abbreviations: UL, uterus lower UU, uterus upper; PL, placenta; FM; fetal membranes; CX, cervix. (B) Total protein was extracted from the lower uterus from mice at GD15, 17, 18, 18.5, 19, or one day postpartum (PP1) and pooled (n=6 per GD). Relative migration of human leukocytes was expressed as the averaged ratio of migrated leukocytes towards each chemoattractant compared to GD15 for each trial. *p<0.05, **p<0.01, ***p<0.001.

FIG. 13. Comparison of intra-uterine chemokines from several mammalian species. Human TL leukocytes are attracted to chemoattractants from cow fetal membranes, cow placental cotyledon (whole), sheep fetal membranes (chorio-allantois), sheep cotyledon (whole), mouse lower uterus at GD 18.5, and human TL fetal membranes (amino-chorion). Control migrations using buffer without tissue protein were about 200 leukocytes in each case and were subtracted from migrations with tissue protein. The amount of tissue protein was the same for each species, 50 μg/chamber. (n=3-6) *p<0.05, **p<0.01, ***p<0.001.

FIG. 14. Representative FACS distribution of mouse leukocytes in LMA using mouse lower uterine chemoattractant during late gestation. The upper left diagram (Total Leukocytes) describes the distribution of GD 15 mouse leukocytes before migration (granulocytes and lymphocytes) plus beads. The other diagrams describe the distribution of leukocytes plus beads that migrate in the LMA during late gestation. The number of granulocytes migrated increases until GD 18.5 and then decreases on GD 19. There is no migration of lymphocytes.

FIG. 15. Effect of IL-1β administration without and with rytvela on GD 16 on mouse lower uterine concentrations of KC (A) and MIP-2 (B) and chemoattractant (C) at GD 17. KC and MIP-2 were estimated using Multiplex. The relative migration of leukocytes was expressed as the ratio of migrated leukocytes towards each chemoattractant compared to GD17 or sham for each trial, then averaged. N=5-7, *p<0.05. * with no bar indicates significance compared to all other groups.

FIG. 16. Term increase and effect of IL-1β alone or with rytvela on cytokine mRNA abundance in peripheral leukocytes in pregnant mice. qPCR values are shown on GD 17 and 18.5 in untreated dams and on GD 17 in dams treated with vehicle, IL-1p alone or with rytvela and rytvela plus vehicle. *p<0.05.

FIG. 17. Effect of IL-1β without or with rytvela on GD 16 on migration of mouse leukocytes from pregnant mice at GD 17 using mouse chemoattractant from GD 18.5 lower uterus (n=3-4). *p<0.05.

FIG. 18 depicts a graph depicting term chemotaxis dependency on either CXCR2 or CCR1. Term leukocyte chemotaxis towards TL homogenized extracts is dependent on either CXCR2 and CCR1 chemokine signaling. Increasing concentrations of SB225002, an inhibitor of CXCR2, or J113863, an inhibitor of CCR1, decrease leukocyte migration.

FIG. 19 depicts preterm in labor chemotaxis is only associated with increased CX3CR1 mRNA expression.

FIG. 20 depicts preterm chemotaxis is dependent on CX3CR1 activity. Preterm leukocyte migration towards preterm laboring homogenized extracts is dependent on CX3CR1 chemokine signaling. Increasing concentrations of the CX3CR1 antagonist, JMS 17-HCl, reduce the number of migrated leukocytes.

FIG. 21 depicts the time required and methods to complete the LMA. The New LMA description considerably reduces the time and cost for performing the assay compared to the Old LMA method.

FIG. 22 depicts a schematic impression of mechanisms involved in leukocyte migration at delivery.

### DETAILED DESCRIPTION

The present disclosure relates generally to migration of human peripheral leukocytes in response to chemotactic factor during pregnancy.

In one aspect, there is provided a method of determining the likelihood of a pregnant subject undergoing delivery within about 7 days

The term “subject” or “patient” or “individual”, as used herein, refers to a eukaryote. A biological sample is typically obtained from a eukaryotic organism including, but not limited to, mammals. Mammalian subjects include, but are not limited to, primates such as a human; non-human primates including chimpanzees and the like; livestock, including but not limited to, cows sheep, pigs, and the like; companion animals, including but not limited to, dogs, cats, horses, rabbits, rodents including mice and rats, and the like.

In a specific example, the subject is a pregnant female human.

In another specific example, the subject is a pregnant female rat.

The term “sample” or “biological sample” as used herein, encompasses a variety of cells, cell-containing bodily fluids and/or secretions as well as tissues including, but not limited to a cell(s), tissue, whole blood, blood-derived cells, plasma, serum, sputum, mucous, bodily discharge, and combinations thereof, and the like. Biological samples may include, but are not limited to, tissue and/or fluid isolated from a subject. Biological samples may also include sections of tissues such as biopsy and autopsy samples, formalin-fixed paraffin-embedded (FFPE) samples, frozen sections taken for histologic purposes, blood and blood fractions or products (e.g., serum, plasma, platelets, red blood cells, white blood cells and the like), sputum, stool, tears, mucus, hair, and skin. Biological samples also include explants and primary and/or transformed cell cultures derived from animal or patient tissues. Biological sample also include fetal membranes, including fetal membranes obtained fragments from the amnion, choriodecidua and whole fetal membranes.

In certain examples, biological samples may also be blood, a blood fraction, urine, effusions, ascitic fluid, saliva, cerebrospinal fluid cervical secretions, vaginal secretions, endometrial secretions, gastrointestinal secretions, bronchial secretions, sputum, cell line, tissue sample, or secretions from the breast.

In a specific example, a biological sample is a blood samples, or a blood fraction.

In another specific example, a biological sample comprises cervical or uterine samples.

A biological sample may be obtained using those methods known to the skilled worker. Methods of obtaining such samples from a subject are known to the skilled worker.

As used herein, “obtaining a sample” or “obtaining a biological sample” refers to such methods as will be well known to the skilled worker. A biological sample may be obtained directly or indirectly from the subject. The term “obtaining” a biological sample may comprise receiving a biological sample from an agent acting on behalf of the subject. For example, receiving a biological sample from a doctor, nurse, hospital, medical center, etc, either directly or indirectly, e.g. via a courier or postal service. In some cases the biological sample is obtained from archival repositories. In one example, the methods of the invention are carried out in vitro or ex vivo.

For example, a blood sample, such as a peripheral blood sample, may be collected using venipuncture.

A biological sample may be obtained by removing a sample of cells from an animal, but can also be accomplished by using previously isolated cells (e.g., isolated by another person, at another time, and/or for another purpose), or by performing the methods described herein in vivo. Archival tissues, such as those having treatment or outcome history, may also be used.

A biological sample can be collected on more than one occasion.

In some examples, a tissue sample may be obtained from a biopsy.

The term “biopsy”, as used herein, refers to the process of removing a tissue sample for the methods described herein, and to the tissue specimen itself. Any biopsy technique known in the art can be applied to the methods of the present invention. The biopsy technique applied will depend on the tissue type to be evaluated, the size and type biopsy, among other factors. Representative biopsy techniques include, but are not limited to, excisional biopsy, incisional biopsy, needle biopsy and surgical biopsy. A biopsy may be a “core-needle biopsy”, or a “fine-needle aspiration biopsy” which generally obtains a suspension of cells from within a target tissue. In the case of placental tissue, biopsies may be conducted pre- or post-delivery. Additional examples include gross apportioning of a mass, microdissection, laser-based microdissection, or other art-known cell-separation methods.

In one example, a biological sample is assessed for leukocyte chemotactic activity, and the number and phenotype of the chemoattracted leukocytes are characterized. Methods of measuring chemotaxis are known, and include the use of a Boyden chamber.

The term “leukocyte” or “white blood cell”, as used herein, refers to any type of white blood cell. Leukocytes may be peripheral leukocytes. Leukocytes include adaptive immune cells and innate immune cells. The term “adaptive immune cells” (or “memory immune cells”) and“innate immune cells” as used herein have their conventional meaning. Examples of leukocytes include, for example granulocytes (e.g., neutrophils, eosinophils, basophils), mononuclear phagocytes, and lymphocytes (e.g., B cells, T cells, natural killer (NK) cells).

Leukocytes may be isolated in accordance with any suitable technique. In one example, leukocytes are isolated from peripheral blood using Polymorphprep™.

Leukocytes may be sorted into particular subcategories or types in accordance with any suitable technique.

The term “Boyden chamber”, as used herein, encompasses generically any instrument used to study chemotaxis (also referred to as chemotactic activity), and in particular trans-membrane chemotaxis. It is also referred to as “trans-well migration” or an in vitro cell migration assay.

The Boyden chamber, is based on a chamber of two medium-filled compartments separated by a microporous membrane. In general, cells are placed in the upper compartment and are allowed to migrate through the pores of the membrane into the lower compartment, in which chemotactic agents are present. After an appropriate incubation time, the number of cells that have migrated to the lower side of the membrane is determined. The Boyden chamber-based cell migration assay has also been called filer membrane migration assay, trans-well migration assay, or chemotaxis assay. A number of different Boyden chamber devices are available commercially, as is well known to the skilled worker. Alternate Boyden chambers may be based on or employ a Boyden chamber whereby the separation of the two (migratory cells- and chemoattractant-containing) units is accomplished not only by a membrane but by cell-coated membranes. The filter membranes may be made from a variety of materials, including, but not limited to polycarbonate, polysulfone, polyvinyl or polystyrene.

The pore size of the membranes selected on the basis of its intended use, and may be in the range for from about 0.5 to about 10 μm diameter, desirably from about 2.5 to about 7.5 μm, or desirably about 3 μm. In a specific example, in the case of leukocytes, the pore size is about 3 μm.

The term “chemoattractant”, as used herein, refers to a molecule or molecules which gives rise to the migration of certain target cells by establishing a chemotactic gradient along which the target cells can move. Examples of chemoattractants include, but are not limited to, a protein(s). Once example of a chemoattractant is a chemokine. One example of a chemoattractant is a cytokine. Chemoattractants may be present in cell and/or tissue extracts. Chemoattractant may be present in a conditioned media obtained from cells and/or tissues and/or extracts.

In some examples, the biological sample is a fetal membrane extract obtained from pregnant human females at term, with or without labour. In other examples, the sample may be amnion, choriodecidua or whole fetal membrane extracts obtained from pregnant human females at term, with or without labour.

The term “normal pregnancy” as used herein refers to a pregnancy the proceeds to term without any complications, and is also referred to as a term pregnancy or full term pregnancy. Typically, a normal pregnancy is a pregnancy without a pregnancy-associated disorder.

As used herein, the term “term pregnancy” and “full term pregnancy” refers to the onset of labour after the 37 week or gestation, in humans. In another example, these terms refer to the onset of labour between about the 37^{th} week and 40^{th} week of gestation.

The term “pregnancy-associated disorder”, as used herein, refers to any condition or disease that may affect a pregnant woman, the fetus the woman is carrying, or both the woman and the fetus. Such a condition or disease may manifest its symptoms during a limited time period, e.g., during pregnancy or delivery, or may last the entire life span of the fetus following its birth. Non-limited examples of pregnancy-associated disorders include preterm labor, preeclampsia, preterm premature rupture of membranes, placental abruption, ectopic pregnancy, fetal chromosomal abnormalities, hypertensive disorders with or without associated proteinuria, chronic hypertension, gestational hypertension (pregnancy induced hypertension (PIH)), and the like.

In one example, a pregnancy-associated disorder is preterm labor (also referred to as preterm delivery), preeclampsia, preterm premature rupture of membranes and/or placental abruption.

In a specific example, the pregnancy-associated disorder is preterm labor.

The terms “preterm labour” and “premature labour” and “preterm delivery”, as used herein, refers to the premature onset of labor resulting in expulsion from the uterus of a viable infant before the normal end of gestation (i.e. pre-term birth or delivery), if not treated. In a specific example, in the case of humans, preterm labour refers to the onset of labor with effacement and dilation of the cervix before the 37th week of gestation. In another example, preterm labour refers to the onset of labor with effacement and dilation of the cervix between about the 20^{th} week of gestation and the 37th week of gestation.

Preterm labour may or may not be associated with vaginal bleeding or rupture of membranes. Preterm labor may or may not be related to factors including without limitation infection (eg, bacterial vaginosis [BV], sexually transmitted diseases [STDs], urinary tract infections, chorioamnionitis), uterine distention (eg, multiple gestation, polyhydramnios), uterine distortion (eg, müllerian duct abnormaities, fibroid uterus), compromised structural support of the cervix (eg, incompetent cervix, previous cone biopsy or loop electrosurgical excision procedure [LEEP]), abruptio placentae, uteroplacental insufficiency (eg, hypertension, insulin-dependent diabetes, drug abuse, smoking, alcohol consumption), stress either indirectly by associated risk behaviors or by direct mechanisms including fetal stress.

The term ‘preeclampsia’, as used herein, refers to a condition that occurs during pregnancy, the main symptom of which is various forms of high blood pressure often accompanied by the presence of proteins in the urine and edema (swelling). Preeclampsia, sometimes called toxemia of pregnancy, is related to a more serious disorder called “eclampsia,” which is preeclampsia together with seizures. These conditions usually develop during the second half of pregnancy (after 20 weeks), though they may develop shortly after birth or before 20 weeks of pregnancy.

The term “placental abruption” as sued herein, refers to a condition that occurs during and may be associated with hypertension, diabetes, a multiply pregnancy, an unusually large amount of amniotic fluid, numerous previous deliveries, loss of material blood, or advanced maternal age

The term “treatment” or “treated”, as used herein, refers to both therapeutic treatment and prophylactic or preventative measures, wherein the object is to prevent or slow down (lessen) the targeted condition or disorder. Those in need of treatment include those already with the disorder as well as those prone to have the disorder or those in whom the disorder is to be prevented.

In one example, the IL-1 receptor (IL-1R) antagonist, rytvela, a 7-amino acid allosteric antagonist of the IL-1R is used in the treatment of pre-term labor.

Treatment for pregnancy-associated disorders are known to the skilled worker.

In some examples, a suitable treatment or medicament is administered for treatment of the pregnancy associated hypertensive disorder, and may for example be selected from aldomet, labatolol, hydralazine, nifedipine (Procardia, Adalat), diuretics, clonidine, calcium channel blockers, vasodilators, magnesium sulphate (MgSO4), and combinations thereof.

In other examples, tocolytic agents that may be utilized in methods as described herein may for example be selected from MgSO4, nifedipine, fenoterol, ritodrine (Yutopar), atosiban, salbutamol, indomethacin, terbutaline (Brethine), oxytocin antagonists, and combinations thereof.

In other examples, steroids that may be used for promoting maturation of foetal lungs include corticosteroids and glucocorticoids (such as betamethasone, dexamethasone, and hydrocortisone).

The term “prediction” and “determination in the likelihood” as used herein, refers to providing a measure of relative risk for developing a pregnancy-associated disorder in a patient. As used herein, the term “providing a prognosis” refers to providing a prediction of the probable course and outcome of a pregnancy-associated disorder.

As used herein, the term “diagnosis” refers to detecting a pregnancy-associated disorder or a risk or propensity for development a pregnancy-associated disorder. It will be appreciated that typically any method of diagnosis includes false positives and false negatives. Accordingly, it is typical that a method of diagnosis does not necessarily provide 100% accuracy.

In one example, a determination of the likelihood of a disorder associated with pregnancy is made by measuring chemotaxis of leukocytes, obtained from a pregnant human female, towards a chemoattractant.

As used herein, the term “diagnosis” refers to detecting a pregnancy-associated disorder or a risk or propensity for development a pregnancy-associated disorder. It will be appreciated that typically any method of diagnosis includes false positives and false negatives. Accordingly, it is typical that a method of diagnosis does not necessarily provide 100% accuracy.

In one example, a determination of the likelihood of a disorder associated with pregnancy is made by measuring chemotaxis of leukocytes, obtained from a pregnant human female, towards a chemoattractant.

A patient identified with a likelihood of preterm labour may receive appropriate treatment, as would be known to the skilled worker.

In accordance with another aspect of the present invention, there is provided a method of monitoring the responsiveness to treatment for disorder associated with pregnancy.

In another embodiment, a method as described herein comprises qualitatively or quantitatively determining, analyzing or measuring a biological sample from a subject for the presence or absence, or amount or concentration, of one or more biomarkers associated with the diagnosis and/or prognosis and/or therapeutic monitoring of pregnancy and/or a disorder associated with pregnancy.

In one example there is provided a method for determining the likelihood of a pregnant subject undergoing term delivery, comprising: obtaining a sample from a pregnant subject, contacting the sample with a reagent to a biomarker, to form a complex between the agent and the biomarker present in the sample; measuring the complex formed to determine the amount or concentration of said biomarker in the sample; wherein an increased likelihood of term delivery is indicated when said biomarker is at a level characteristic of a full term pregnancy.

In one example, the biomarker is CXCR2, CXCR3, CXCR5, CCR1, CCR3, CCR5, or CCR7.

The term “prognostic marker” or “biomarker” as used herein refers to a marker that informs about the outcome of a patient in the absence of systemic therapy or portends an outcome different from that of the patients without the marker, despite empiric (not targeted to the marker) systemic therapy.

The term “prognosis” as used herein, refers to the prediction of the likelihood a pregnancy associated disorder in a specific example, the disorder is premature delivery.

The term “therapeutic monitoring” as used herein refers to the observation of the response of the subject to the treatment administered to it.

The determination, analysis or measurement of the biomarker is correlated with normal term pregnancy, or a disorder associated with pregnancy. In some examples, a patient sample is compared to a control sample. In some examples, a control is not used and qualitative or quantitative methods are used to determine the presence or absence, or amount or concentration of the protein of interest.

In one example, in determining whether there is strong, weak or minimal (or absent) amount of the biomarker, the patient sample may be compared to one or more control samples. In one example, a control sample has had known and/or established level of the biomarker. In one example, a control sample is a patient sample that has known and/or established levels of biomarker expression and/or known clinical outcome. In one example, a control is a cell line that has a known amount of biomarker expression. In another example, a control sample is a sample from a full term pregnancy.

The term “expression”, as used herein, and for example in reference to a biomarker such as CXCR2, CXCR3, CXCR5, CCR1. CCR3, CCR5, CX3CR1, or CCR7, refers to al indicators of transcriptional expression of the biomarker encoding gene. Such indicators include biomarker transcript products, generated as a result of transcription of the biomarker gene; translation products, including al forms of the biomarker protein, generated as a result of translation of the biomarker transcripts; and demonstrable or otherwise measurable biomarker activity.

As used herein, “biomarker protein”, includes, but is not limited to, full-length proteins, mature proteins, pre-proteins, polypeptides, isoforms, mutations, variants, post-translationally modified proteins and variants thereof. Biomarker protein detection is know to the skilled worker, and is discussed herein.

Biomarker transcripts or mRNA can be measured using any of many techniques known to those of skill in the art, including, but not limited to, northern hybridization, PCR, reverse transcription followed by PCR, quantitative real-time PCR, nuclease protection assay, and in situ hybridization.

Biomarker activity can be measured by a variety of assays known to those of skill in the art. A suitable method can be selected to determine the activity of proteins encoded by the biomarker genes according to the activity of each protein analyzed. For biomarker proteins, polypeptides, isoforms, mutations, and variants thereof known to have enzymatic activity, the activities can be determined in vitro using enzyme assays known in the art. Such assays include, without limitation, protease assays, kinase assays, phosphatase assays, reductase assays, among many others. Modulation of the kinetics of enzyme activities can be determined by measuring the rate constant K_{M} using known algorithms, such as the Hill plot, Michaelis-Menten equation, linear regression plots such as Lineweaver-Burk analysis, and Scatchard plot.

Biomarker protein can be measured/detected by a variety of techniques known to the skilled worker, including, but not limited to, immunoassays using a biomarker specific antibody. Protein levels can also be determined using a specific antibody or mass spectroscopy in conjunction with 2 dimensional gel electrophoresis (separation of proteins by their isoelectric point (IEF) In the first dimension followed by molecular weight determination using sodium dodecyl sulphate polyacrylamide gel electrophoresis (SDS-PAGE)).

In other examples, a biomarker protein is detected using a binding agent (also referred to an as agent) including, but not limited to, a lectin, nucleic acid (e.g. DNA, RNA), monoclonal antibody, polyclonal antibody, Fab, Fab′, single chain antibody, synthetic antibody, aptamer (DNA/RNA), peptoid, zDNA, peptide nucleic acid (PNA), locked nucleic acid (LNA), synthetic or naturally occurring chemical compound (including but not limited to a drug or labeling reagent), dendrimer, or any combination thereof. In some instances, a single agent is used to detect a biomarker. In other instances, a combination of different agents is used to detect a biomarker.

Detection includes direct and indirect detection. Similarly, a binding agent can be directly or indirectly labeled.

The quantity of one or more biomarkers can be indicated as a value. The value can be one or more numerical values resulting from the evaluation of a sample, and can be derived, e.g., by measuring level(s) of the biomarker(s) in a sample by an assay performed in a laboratory, or from dataset obtained from a provider such as a laboratory, or from a dataset stored on a server.

In some examples, qualitatively or quantitatively determining, analyzing or measuring a biological sample from a subject for the presence or absence, or amount or concentration, of one or more prognostic marker associated, is carried out using antibodies to the biomarker.

The term “antibody” and “antibodies” includes, but is not limited to, monoclonal and polyclonal antibodies. Antibodies may be derived from multiple species. For example, antibodies include rodent (such as mouse and rat), rabbit, sheep, camel, chicken, and human antibodies. In another example, antigen binding fragments which specifically bind to PDGFRα are used. In some example, the antibodies also comprise a label.

The term “label” as used herein is an identifiable substance that is detectable in an assay and that can be attached to a molecule creating a labeled molecule. The behavior of the labeled molecule can then be monitored and/or studied and/or detected.

Examples of labels include, but are not limited to, various enzymes, prosthetic groups, fluorescent materials, luminescent materials, bioluminescent materials, radioactive materials, positron emitting metals using various positron emission tomographies, and nonradioactive paramagnetic metal ions. The detectable substance may be coupled or conjugated either directly to the antibody (or fragment thereof) or indirectly, through an intermediate. The particular label used will depend upon the type of immunoassay. Antibodies can be tagged with such labels by known methods.

The term “binds specifically” refers to high avidity and/or high affinity binding of an antibody to a specific polypeptide e.g., an epitope of for the biomarker protein. Antibody binding to its epitope on this specific polypeptide is stronger than binding of the same antibody to any other epitope, particularly those which may be present in molecules in association with, or in the same sample, as the specific polypeptide of interest. Antibodies which bind specifically to a polypeptide of interest may be capable of binding other polypeptides at weak, yet detectable, level. Such weak binding, or background binding, is readily discernable from the specific antibody binding to the compound or polypeptide of interest, e.g., by use of appropriate controls, as would be known to the worker skilled in the art.

The methods of the present invention may be accomplished using any suitable method or system of immunohistochemistry. Non limiting examples include automated systems, quantitative IHC, semi-quantitative IHC, and manual methods.

The term “quantitative” immunohistochemistry refers to an automated method of scanning and scoring samples that have undergone immunohistochemistry, to identify and quantitate the presence of a specified biomarker, such as an antigen or other protein. For example, to quantitate for the biomarker protein, the score given to the sample is a numerical representation of the intensity of the immunohistochemical staining of the sample, and represents the amount of target biomarker present in the sample. As used herein, Optical Density (OD) is a numerical score that represents intensity of staining as well as the percentage of cells that are stained.

Automated sample processing, scanning and analysis systems suitable for use with immunohistochemistry are known in the art, and may be used with the methods described herein. Such systems may include automated staining and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples.

Other examples that may be used in the detection, analysis or measurement of for the biomarker protein include, but are not limited to, immunoprecipitation, immunoblotting, mass spectrometry, quantitative fluorescence activated cell sorting, enzyme linked immunosorbent assay, immunohistochemistry, quantitative immunohistochemistry, fluorescence resonance energy transfer, Forster resonance energy transfer, and biomolecular fluorescence complementation. Additional examples are as described herein.

It will be appreciated that in some circumstances, a patient which is initially identified a not having an increased likelihood of developing a pregnancy associated disorder, may relapse or reoccur. The methods as described herein may be used in the therapeutic monitoring of a patient, to monitor and identify those patients which may later develop a pregnancy associated disorder.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLES

### Example 1

### Abstract

Leukocyte invasion of the human uterus occurs at term and preterm to activate it for labor. A proxy for this phenomenon is to examine peripheral leukocyte migration ex vivo. We observed the pattern and regulation of leukocyte migration during human pregnancy with our leukocyte migration assay (LMA). Leukocyte migration to a term labor (TL) human fetal membrane (hFM) chemoattractant was slightly higher in the second trimester than first or third trimesters, increased at term delivery and decreased postpartum. Migration increased dramatically from 35 weeks to TL. Preterm labor leukocytes migrated better to preterm hFM chemoattractant than term leukocytes. Exposing leukocytes to conditioned medium from TL hFM explants or serum from TL women increased migration. Interleukin (IL) 1-β stimulated chemoattractant output from leukocytes. A number of chemokines increase their concentrations in maternal serum with labor onset at term, and several chemokine receptors and their intracellular mediators increase leukocyte expression in relation to migration at term. These data indicate that the hFM chemoattractant is different at term and preterm, it conditions peripheral leukocytes for migration through the expression of specific chemokine receptors and intracellular signaling pathways. IL-1 β-stimulated leukocyte chemokine release enhances migration. The LMA is a robust assay for assessing leukocyte migration.

### Introduction

Leukocytes invade the human uterus throughout pregnancy for various purposes to facilitate implantation, pregnancy maintenance and termination, uterine involution and postpartum cervical remodeling (1-3). A specific body of research evidence suggests that peripheral circulating leukocytes invade human and animal intrauterine tissues at term and preterm birth (PTB) to effect labor. In the human myometrium and decidua, neutrophils, macrophages, and T-lymphocytes invade during term labour (TL) (4-7), and in the cervix, leukocytes infiltrate the tissue at term (8-11). In mice both full- and sub-PTB-inducing doses of the gram-negative bacterial mimic, lipopolysaccharide (LPS), were sufficient to stimulate neutrophil infiltration of fetal membranes (12). This invasion was blocked with rytvela, an interleukin (IL)-1 receptor antagonist indicating a critical, upstream role for IL-1β (12). Once these leukocytes invade, it has been proposed that they release an array of matrix metalloproteases, prostaglandins, cytokines, chemokines and other effectors that not only amplify this inflammatory event, but also facilitate the uterine transition from pregnancy to labor through activation of the uterus for labor (e.g. altered expression of uterine activation proteins, remodeling of the cervical extracellular matrix, and breakdown of the fetal membranes), which culminate in parturition (13, 14).

We have studied the mechanisms of leukocyte invasion of uterine tissues at term and preterm in pregnant women. We identified that the human fetal membranes (hFM), amnion and chorion, release a chemoattractant whose levels increase as term labor approaches, and the responsiveness of the leukocytes to the chemoattractant also increases as labor approaches (15, 16). We developed a leukocyte migration assay (LMA) using a Boyden chamber that mimics some of the in vivo actions of leukocyte extravasation to study this phenomenon in rats, guinea pigs, and humans at term and preterm (17-19).

These observations provoke several questions in pregnant women including what is the pattern of peripheral leukocyte attraction to term hFM chemoattractant throughout pregnancy, is the hFM chemoattractant similar at term and preterm, what are the changes that occur in leukocyte biology that associate with migration activation and does the upstream mediator, IL-1β, affect either hFM chemoattractant or leukocyte migration. Evaluation of longitudinal changes in leukocyte migration could demonstrate whether it increases gradually or suddenly from early pregnancy to labor at term and preterm. These data would inform whether the LMA in its current form could be a potential diagnostic to predict PTB risk.

The purpose of this study was to explore changes, both in vivo and ex vivo, in peripheral leukocyte and hFM biology during pregnancy and at term and preterm delivery in pregnant women.

### Results

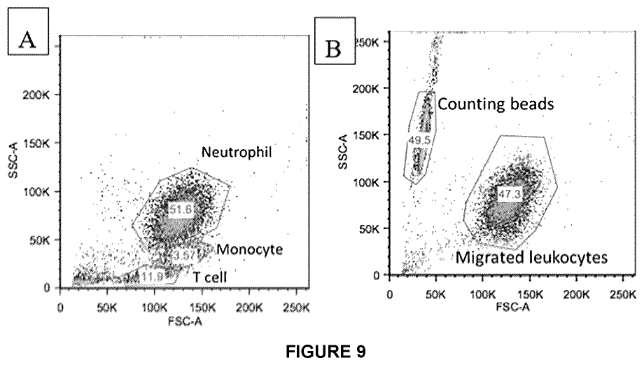

Migration of peripheral pregnant leukocytes in a Boyden chamber. FIG. 9 is a flow cytometry analysis of the relative proportion of isolated leukocytes from whole blood in a typical LMA in the upper chamber (A) and the cells that migrated across the filter (B). A subset of samples was measured by flow cytometry using antibodies (CD11b, CD14. CD3, and CD4). Approximately 99% of migrated leukocytes were neutrophils (CD11b+), which were detected by setting specific gates for each leukocyte population based on dot-plot graphs of side-scatter (SSC) versus forward scatter (FCS) or detecting cells positive for CD11b+ (FIG. 98).

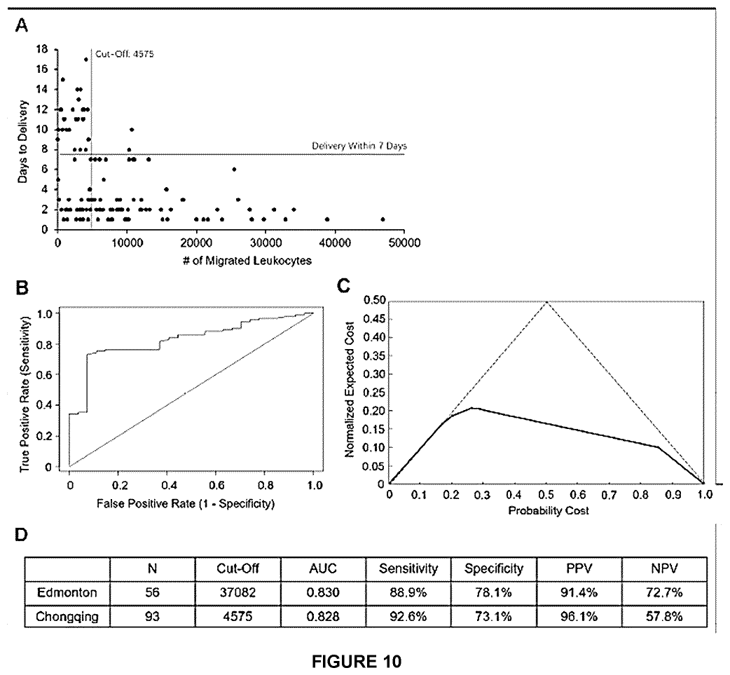

FIG. 10 describes the performance of the LMA. The cutoff for predicting delivery within 7 days is 4575 cells using our own 3D printed chambers as compared to 37,082 cells in a separate publication from our team using commercial chambers (FIG. 10A) (19). The area under the curve is 83%, and the cost-curve plot of the migration data demonstrates that the probability-cost function (X axis) produces the lowest error rate (Y axis) when the probability cost is 25% to 85% (X axis) (FIG. 10B, 10C). The performance of the LMA is compared between two jurisdictions with two different operators and commercial chambers vs. those of our own manufacture (Table, 2D). The LMA is a very robust assay.

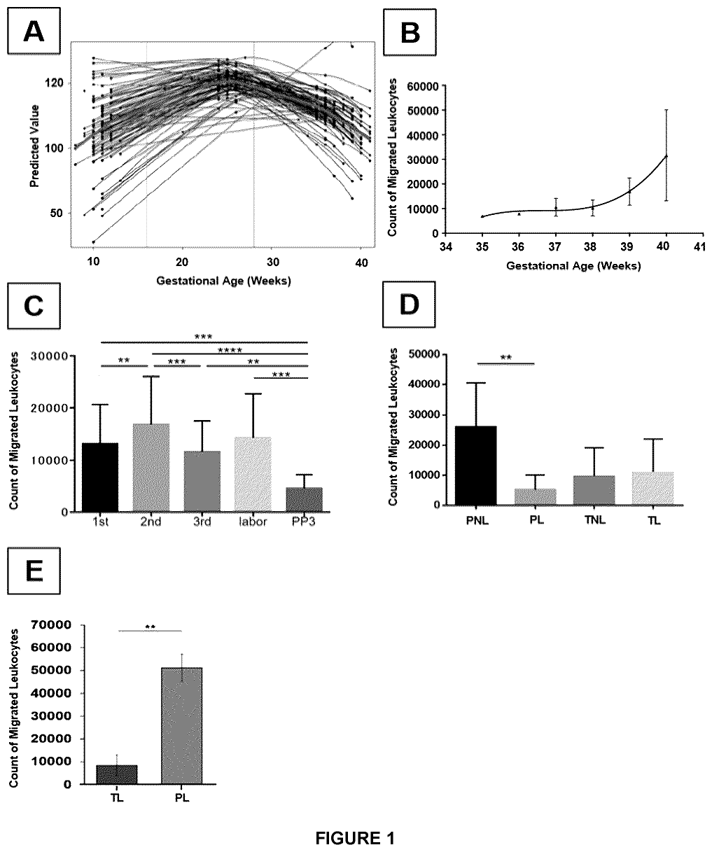

Longitudinal changes in leukocyte migration throughout pregnancy. FIG. 1A, B describes the longitudinal changes in migration of leukocytes from women throughout pregnancy to TL hFM extract chemoattractant or during the last five weeks of pregnancy. In FIG. 1A, the longitudinal LMA data were tested for normality and were found to not be normally distributed. Thus they were transformed by taking the square root, which led to a normal distribution. The quadratic regression model was used to fit an upward convex curve to the data, y=74.78+3.65GAW−0.07GAW² where GAW=gestational age in weeks. It is evident that while individual fluctuations throughout pregnancy were large, the overall tendency was for values to increase slightly in the middle of pregnancy, and then decrease in the latter half of pregnancy.

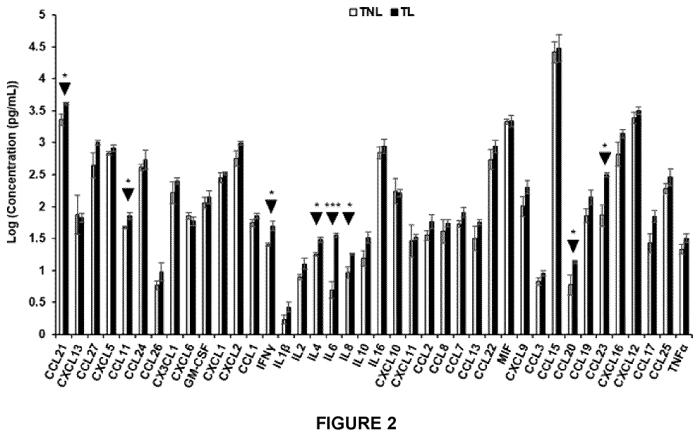

Studying the LMA from 35-40 weeks and those in labor at term, the data indicated a sharp increase in leukocyte in serial samples in late gestation (r²=0.4566, p<0.01) (FIG. 2B). But when organized by trimester, the migration of leukocytes from the first and third trimesters was lower than that of the leukocytes from the second trimester (p<0.01) (FIG. 1C), and while there appears to be an increase in the mean levels of migration from the third trimester to labor at term, this was not significant. The migration of leukocytes isolated at postpartum day 3 was dramatically and significantly lower (p<0.001) than that of leukocytes from each of the three trimesters and at term labor (FIG. 1C). These data suggest that the presence of the placenta and tissues of pregnancy influence peripheral leukocyte migration to term hFM chemoattractant. When organized by event and not trimester, there was higher migration of leukocytes at PNL (composed of both second and third trimester normal pregnancies) than at PT (p<0.01) or TNL or TL (NS) when using term hFM chemoattractant (FIG. 10).

These data suggest that TL hFM chemoattractant may not be an appropriate match for earlier gestation leukocyte chemoattraction. To test this possibility, hFM from PL deliveries was extracted for chemoattractant and tested against TL and PL leukocytes (FIG. 1E). There was considerably greater chemoattraction of PL leukocytes than TL leukocytes to PL chemoattractant (p<0.01). These data suggested the chemoattractant might be controlling the leukocyte phenotype and migration. We therefore started by testing whether there was an increase in chemokines and cytokines in maternal serum with term labor onset and what this could mean for leukocyte activation.

We examined maternal TNL and TL serum for changes in cytokine and chemokine concentrations using multiplex and concluded that a number of cytokines and chemokines significantly increase their concentration in maternal serum with term labor onset: CCL11, CCL20, CCL21, CCL23, IFN-γ, IL-4, IL-6, and IL-8 (CXCL8) (FIG. 2). Both CCL2 and CCL7 increased with labor onset, but these were not significant. In combination, these may contribute to leukocyte activation and migration.

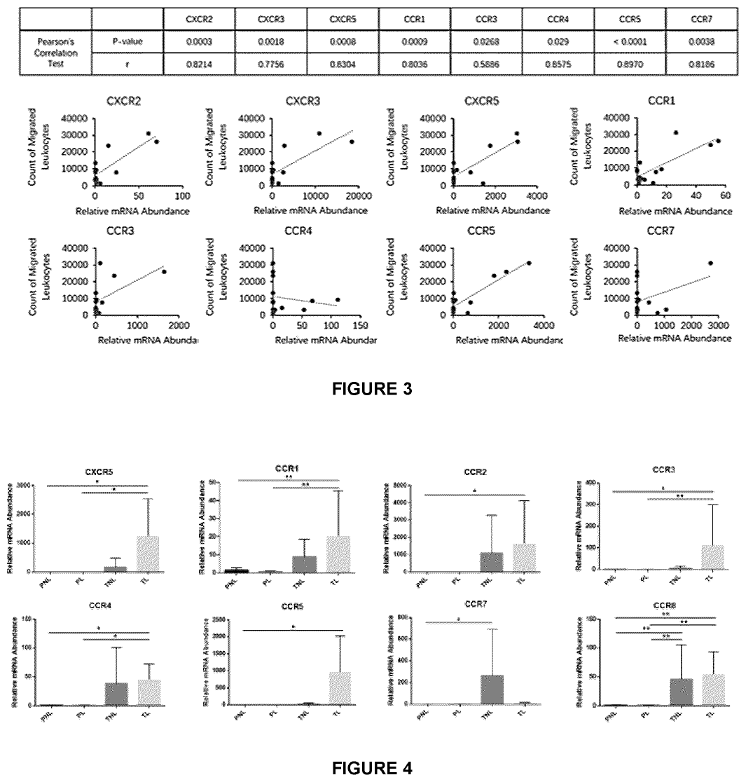

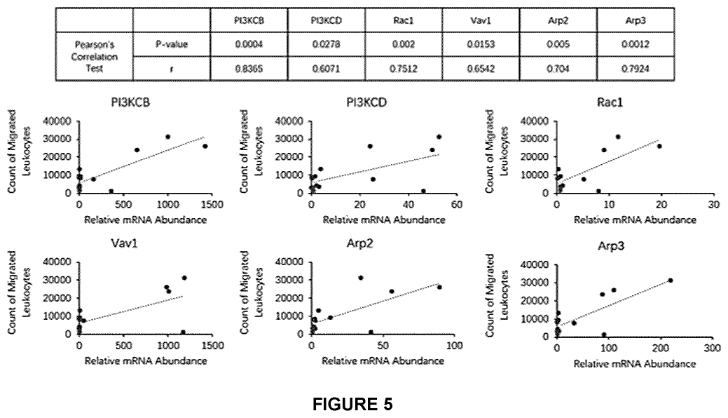

Testing leukocytes for phenotypic expression of chemokine receptors. We next compared the expression of chemokine receptors by leukocytes with their migration. FIG. 3 demonstrates chemokine receptor mRNA expression correlated with migration. Seven of the eight chemokines tested increased expression significantly in response to migration; only CCR4 mRNA expression was negatively coordinated with migration to TL hFM chemoattractant. We then compared expression of the chemokines at the four different stages of pregnancy. PNL, PL, TL and TNL (FIG. 3). The eight chemokines tested all showed a significant increase in expression between PNL and TNL or TL. Most chemokine receptors demonstrated an increase in expression between TNL and TL (NS). These eight chemokine receptors were all associated with term leukocyte activity as there was no or little mRNA abundance at PNL or PL. In conjunction with chemokine receptor expression, we observed the correlation of mRNA expression of six different intracellular signaling molecules with migration in TL leukocytes as shown in FIG. 5. These signaling molecules all facilitate F-actin polarization in neutrophils. We therefore examined neutrophils for F-actin distribution from PNL, TNL and TL pregnant women. There was a clear increase in the percentage of neutrophils with polar localization of F-actin in TL women (p<0.05). This information correlates well with increased expression of signaling molecules and migration.

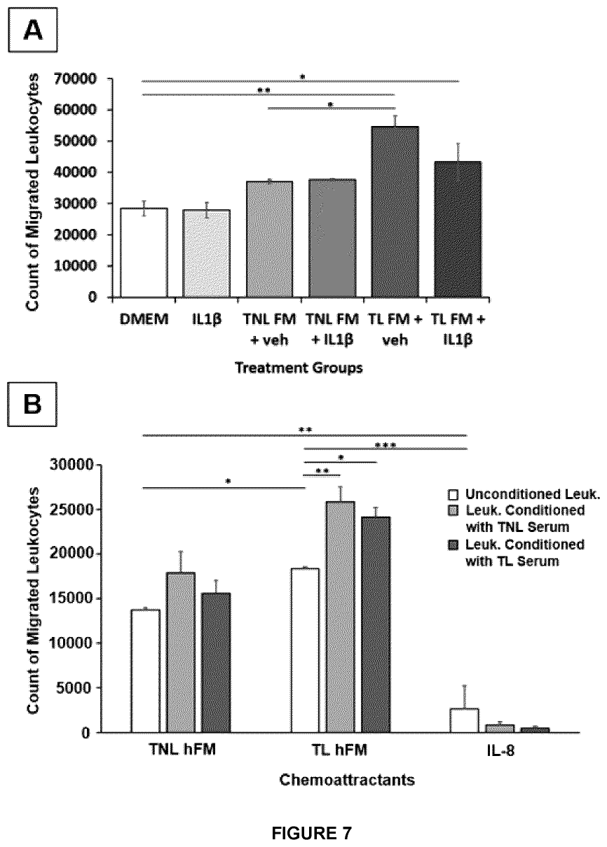

TL hFM conditioned medium increases TNL leukocyte response to TL hFM chemoattractant. The data from FIG. 1 suggest that the placental tissues affect peripheral leukocyte migration in response to TL hFM chemoattractant. Given the correlation between increased chemokines in maternal TL serum with increased polarization of F actin, increased pathways for this polarization and increased expression of chemokine receptors with increased migration, we decided to test whether term hFM explant conditioned medium and maternal TL serum treatments could alter leukocyte migration. We additionally tested whether IL-1β would accelerate chemoattractant output or leukocyte migration, which we found to be the case in mouse experiments. We tested whether hFM conditioned media without or with IL-1β co-treatment could modify migration of peripheral leukocytes by pretreating TL and TNL hFM explants with IL-1β or vehicle for 6 h after which these single conditioned media were collected and then incubated with TNL leukocytes for 1 h. Following this treatment, the leukocytes were washed and resuspended in RPMI for assessment of migration to hFM chemoattractant using the LMA. FIG. 7A shows that pretreating TNL leukocytes with TL hFM explant conditioned medium increased their migration to TL chemoattractant. Treatment of hFM explants with IL-1β had no effect on leukocyte sensitivity to migration.

The next logical step was to question whether late gestation maternal serum could increase leukocyte migration to term chemoattractant (FIG. 7B). Leukocytes obtained from TNL women were exposed to serum from TNL and TL women for 1 h and then tested for migration to either TNL or TL chemoattractant. Both TNL and TL serum incubation significantly increased TNL leukocyte migration in response to TL chemoattractant. These data suggest that the fetal membranes secrete factors into maternal serum that increase leukocyte responsiveness to hFM chemoattractant.

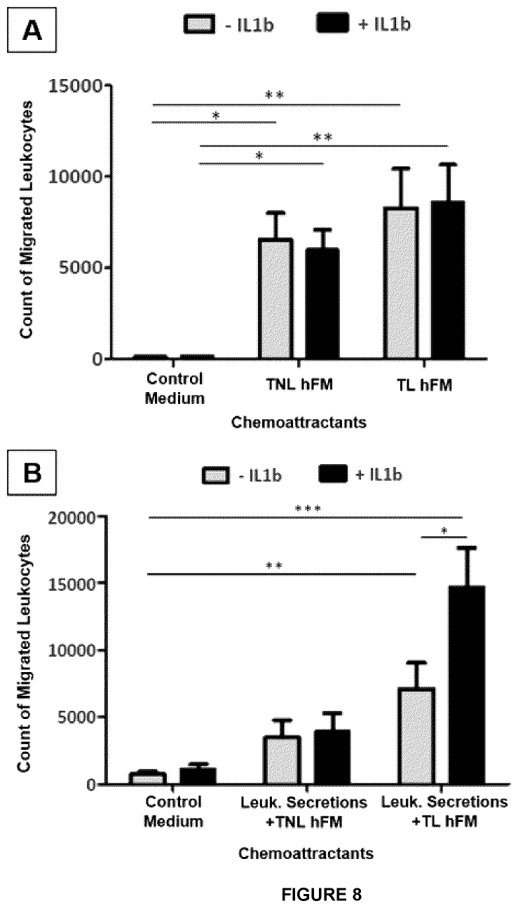

Further tests of whether IL-1β could directly stimulate chemoattractant from hFM explants were performed. We learned that explants released more chemoattractant with labor onset at term compared to term not in labor hFM, but IL-1β did not promote more chemoattractant release (FIG. 8A). Conversely, when treated with hFM conditioned medium, TL peripheral leukocytes from pregnant women secrete chemotactic factors that stimulate considerable migration from fresh TL leukocytes. In this case, IL-1β treatment of the leukocytes plus hFM explant conditioned medium leads to greater leukocyte chemoattractant release (FIG. 8B). Taken together, the effect of IL-1β may be directed towards altering leukocyte biology and not fetal membrane biology

### Discussion

Leukocyte invasion of uterine tissues (myometrium, decidua, fetal membranes and cervix) at term and preterm labor is a well-described phenomenon in several species (1-7, 9-11). Our LMA is a sensitive, precise and robust bioassay that assesses leukocyte (primarily granulocytes, 99%) migration through a 3 μm filter pores toward the hFM chemoattractant in the lower Boyden chamber (FIG. 9, 10). In this study, our hypothesis tested whether leukocyte migration remained low during pregnancy and then rose dramatically in relation to term delivery. Our hypothesis was proven wrong since term hFM chemoattractant attracted leukocytes in the LMA throughout gestation with a small increase in the second trimester (FIG. 1A). There was a non-significant increase, though, when all term labor assays were compared to third trimester assays; and then a significant decrease occurred 3-4 days postpartum (FIG. 1C). This decrease was likely due to the expulsion of the products of conception, the source of the chemoattractant (15, 16). However, when we longitudinally examined assays from four participants from 35 to 40 weeks gestation, we observed a significant increase in migration (FIG. 1B). The PNL cells (FIG. 1D), which would most resemble second trimester and third trimester pregnant women, were more attracted to term chemoattractant than TL cells. But there were fewer PL cells attracted to TL chemoattractant than TL cells (1D). Conversely the chemoattractant from PL hFM is much better at attracting PL leukocytes than TL leukocytes. These data suggest the chemoattractants at TL and PL are different and that a gestational-age matched hFM chemoattractant should be used to assess leukocyte migration during pregnancy.

Nearly all species of leukocytes invade the uterus at one time or another during pregnancy (1). Granulocytes, particularly neutrophils, as well as monocytes in peripheral blood increase in number during pregnancy (20, 24-26) and show functional changes relevant for pregnancy: they increase in numbers due to stress and endocrine and inflammatory stress mediators (27, 28); they decrease their rate of apoptosis (29); and they modulate macrophage phenotypes by suppressing their pro-inflammatory cytokine release (30, 31). Since we observed an increase in neutrophil migration during the last five weeks of pregnancy (FIG. 1B, FIG. 10), the possibility exists that hFM chemoattractant activates granulocytes for migration and uterine invasion in the days and weeks before delivery (1, 20). Our data (FIGS. 1B, D, E) suggest that the nature or composition of the hFM chemoattractant changes according to the nature of the labor, PL or TL. But equally intriguing is the possibility that late in normal gestation increasing hFM chemoattractant ‘matures or primes’ circulating leukocytes for migration)—a process which might include expression of chemokine receptors (20). We tested this possibility.

Our data support the likelihood that TL hFM chemoattractant activates peripheral leukocytes for migration and uterine invasion. First, we demonstrated an increase in circulating levels in maternal serum of eight chemokines and cytokines with labor onset (FIG. 2). There may be others that also increased that were not included in our multiplex plate. We then found that there was a significant positive correlation between the mRNA abundance of seven chemokine receptors and migration in TL leukocytes. All of these receptors demonstrated measurable mRNA abundance in term samples (TNL or TL), and most showed increases between TNL and TL labor, whereas their abundance was not or barely detectable at PNL and PL labor. This indicates that the phenotype of leukocytes at TL is considerably different than it is at PNL or PL.

The fact that several chemokines, pro-inflammatory cytokines, and even an anti-inflammatory cytokine (IL-4) increase their concentration in maternal peripheral plasma in relation to term delivery (FIG. 2), suggests that a significant source of these mediators is from the hFM. It is highly likely that a combination of chemokines compose the chemoattractant produced by the hFM. For many years, Van Damme and his colleagues studied neutrophil chemoattraction to various chemokines and learned that in combination they were far better chemoattractants than they were individually due to synergistic effects (32, 33). The data from FIGS. 3 and 4 showing a correlation between expression of seven chemokine receptors and TL leukocyte migration and that expression increases only at term and not earlier in pregnancy (e.g. PNL or PL) suggests that the expression of chemokine receptors (34) is quite different at PL.

Neutrophil polarization, which is an important component of neutrophil chemotaxis (34), Involves PI3K- and Rac-dependent actin polymerization at the leading edge of the cell in response to chemoattractants (38). Neutrophils express all four known class I PI3K isoforms (expressed by PIK3CA/BID/G), all of which, when activated, generate a chemical gradient of PIP3 that generates directional actin polymerization (39). Whether PIK3CB is essential for neutrophil chemotaxis is not fully understood, but PIK3CD is important for neutrophil directional movement (40). The small GTPase Rac1, on the other hand, is a well-characterized regulator of the Arp2/3 complex that is involved in branched actin filament polymerization at the leading edge (38). Defective polarization and directionality are apparent in the neutrophils of Rac1−/− mice (41). Rac1 is also involved in a positive feedback loop with PI3K lipid products at the leading edge of neutrophils (42). Lastly, Vav1 has been demonstrated to be essential for neutrophil migration out of the vasculature because it induces cytoskeletal reorganization in response to neutrophil adhesion via LFA-1 (43). The demonstration of increased F-actin polarity in TL neutrophils is an early step in extravasation that precedes migration and uterine invasion (Filippi, 2019).

Interestingly, IL-1β pretreatment of TNL or TL hFM explants had no effect on the secretion of chemoattractant (FIG. 8A). However, when TNL leukocytes were pretreated with IL-1β, they released chemoattractant into the conditioned medium (FIG. 8B). These data are consistent with our mouse data whereby IL-1 β treatment, in vivo, did not stimulate an increase in chemoattractant from the uterus, but it did stimulate enhanced leukocyte migration. Such data are consistent with the hypothesis that IL-1 β is the mediator of silent inflammation in the pregnant uterus that leads to the amplification of uterine activation by pro-inflammatory cytokines and ultimately to delivery at term and preterm (13, 14, 21, 22).

In conclusion, our data show that TL chemoattractant is different from PL chemoattractant. Future studies will examine PL chemoattractant in more detail. Our data also support the concept that chemoattractant from hFM at term is a critically important regulator of leukocyte activation and migration that accelerates the processes of labour. While hFM chemoattractant is not stimulated by IL-1 β, leukocyte chemoattractant is. This suggests that the mechanisms of leukocyte activation and migration differ between term and preterm labor. Since the amnion and chorion are fetal tissues, the data suggest that the fetus determines the timing of its delivery through the expression of chemokines from these tissues.

### Methods

In Vivo Studies—Longitudinal Examination of Leukocyte Migration During Pregnancy

Patients. This study was approved by the local ethics committee of Juntendo University (Tokyo, Japan), Faculty of Medicine (No. 13-131). One hundred women were recruited for the longitudinal study with peripheral blood collection. All participants provided written, informed consent to be included in the study prior to enrolment which included publication of the anonymized results. Patients with normal pregnancies having no medical or obstetrical complications were included. Pregnant women who had multiple pregnancy, fetal congenital disease, and clinical signs of infection (body temperature over 38° C.) were excluded. Blood was drawn three times at an outpatient clinic, once in each trimester, when patients had a health checkup during the 1st trimester (8-13 weeks of gestation), 2nd trimester (24-28 weeks of gestation), and 3rd trimester (34-37 weeks of gestation). Blood was collected twice more when patients were admitted to hospital for labor starting with the first stage of delivery and on postpartum day 3 or 4. Blood was drawn in the 2nd trimester at the same time when 50 g glucose tolerance test was performed. In order to determine whether the oral glucose test affected the LMA, it was examined before and after intake of sugared water and shown to have no effect on the migration of leukocytes (data not shown).

Fetal membrane extracts. Fetal membranes from women who underwent spontaneous vaginal delivery at term were collected according to published protocols (15, 19). Exclusion criteria were the same as for leukocyte collection. Immediately after delivery, fetal membranes were cut into 5 cm square pieces and washed three times with 1× phosphate buffered saline (PBS) to remove blood and debris. The pieces were cut into smaller pieces and homogenized with 2 mL of Dulbecco's Modified Eagle's Medium (DMEM). They were then centrifuged at 3,000×g for 30 min at 4° C. followed by 20,000×g for 1 h at 4° C. Supernatants were collected and pooled, and protein concentrations were adjusted to 4 μg/μL in each sample using the bicinchoninic acid method. Each sample was examined for chemoattractant activity toward leukocytes using LMA. Fetal membrane extracts from 80 women were mixed together, aliquoted into 100 μL samples, and stored at −80° C. before use in the LMA.

Blood sampling and leukocyte isolation. Peripheral blood samples were collected from pregnant women. Four milliliters of peripheral blood samples were collected in tubes with heparin coating, and 0.8 mL HetaSep (Stemcell. Tokyo, Japan) was added. Solutions were mixed well, and the tubes were placed in an incubator at 37° C. for 10 min to allow the sample to settle until plasma separation. The leukocyte rich plasma layer was harvested, placed in a 50 mL tube, and washed with a 4-fold volume of 1×PBS. The sample was centrifuged at 120×g for 10 min at 20° C. without stopping to remove platelets. The supernatant was removed, and leukocytes were resuspended in 4 mL Hyclone™ Roswell Park Memorial Institute 1640 medium (RPMI) (SIGMA-ALDRICH, Co., MO, USA) containing 2.0 mM L-glutamine and NaHCO₃. KOVA Glasstic Slide 10 With Counting Grids (KOVA International Company, Biochemical Diagnostic Inc., CA, USA) were used to count leukocytes. The number of dead leukocytes was recorded using the Trypan blue method and the suspension mixture was only used when the viability rate was greater than 95%. The leukocyte suspension was diluted using RPMI to a final concentration of 1×105 cells/50 μL and was used in the LMA within an hour of isolation.

Leukocyte migration assay (LMA). The LMA was carried out with the Modified Boyden chemotaxis chambers (AP48; Neuro Probe, Gaithersburg, MD, USA) as we published (19). The chamber is divided into the upper and lower berths. Twenty-five microliters of fetal membrane extracts were placed in the lower chamber of each well. DMEM, as a negative control, was placed in the lower chamber. A polycarbonate membrane with 3 μm pores (Neuro Probe, Gaithersburg. MD, USA) was next placed over the lower chamber, followed by a rubber gasket. Then 100,000 isolated leukocytes from whole blood of pregnant women were placed in the upper chamber of each well. The chambers were incubated for 90 min at 37′C in a humidified air incubator containing 5% CO₂. After incubation, migrated leukocytes were removed from the lower chamber and put into 5 mL BD Falcon™ polystyrene round bottom tubes (Thermo Fischer Scientific K.K., Yokohama, Japan). Three hundred microliters of OptiLyse (Beckman Coulter, Beckman Coulter, Inc., Brea, CA, USA) were added to lyse RBCs, and the sample was vortexed immediately for 1 sec. The tube was incubated for 15 min at room temperature (20-25° C.) while protected from light. One microliter 1×PBS was added to each tube. Following centrifugation at 1,000×g for 10 min at 4′C, the supernatant was removed and 475 μL 1×PBS with 1% formalin was added to fix the leukocytes. The lower chamber samples were then kept covered in a 4′C cold room until quantification using flow cytometry (BD FACS Verse, BD Biosciences, Tokyo, Japan).

Flow cytometry. The number of leukocytes that migrated from the upper chamber to the lower chamber through the polycarbonate membrane was counted using flow cytometry. CountBright™ Absolute Counting Beads (25 μL, Life Technologies, Tokyo, Japan) were added to the lower chamber samples and analyzed using flow cytometry. Leukocytes were run using a BD FACS Verse (BD Biosciences) with CountBright™ Absolute Counting Beads (Life Technologies). At least 10,000 events were collected for al cells and data were saved for later analysis on FLOWJO (LLC, Ashland, OR, USA). Leukocytes were gated based on their forward and side scatter. The total number of leukocytes that migrated was counted using the beads added to the tube. The number of leukocytes that migrated in the blank (negative control) wells containing DMEM (ca. 50-100 cells) was subtracted from all samples in the final calculations.

Statistical analysis. Data were analyzed and graphs were drawn using GraphPad Prism Version 5.0 software (GraphPad Prism Software, CA, USA). Data were tested for normal distribution and significance was determined with an unpaired t test for two group comparisons. One-way ANOVA followed by Tukey's multiple comparison post hoc test was used to detect statistically significant differences between the study groups when comparing more than two groups. A p value <0.05 was considered significant. Data are shown as the mean±SD and in the statistical analysis, the normality of samples was examined, and the square root transformation was applied to the data to alleviate deviation from normal distribution if required. At least the three last time points of pregnancy (initiation, midterm and late term) were used to evaluate the time change of counts of migrated leukocytes. The data were analyzed using ANOVA in SAS (Version 9.1; SAS Institute, Cary. NC, USA) for repeated measures. We investigated two models, one was a simple linear model for the pregnancy duration and the second model was a quadratic model for the square of the labor duration.

Ex vivo studies—adaptive changes in chemoattractant and leukocyte responsiveness during pregnancy.

This segment was led at the University of Alberta and Chongqing Medical University by the same operator (HL). In each case, it received ethical approval by the relevant REB, and participants provided informed, written consent. Human whole blood and hFM were collected from preterm not in labor (PNL 28-36 weeks), preterm in labor (PL, 28-36 wk), term not in labor (TNL) and term in labor (TL) pregnant women at the Royal Alexandra Hospital (Edmonton, AB, Canada) and the First Affiliated Hospital (Chongqing, PR China). Labor was documented by a cervical dilation of greater or equal to 4 cm in the presence of uterine contractions. Women with clinical infection, premature rupture of membranes, diabetes mellitus, immunological problems, non-singleton pregnancies, intrauterine growth restriction, preeclampsia or dysfunctional labor, and recipients of progesterone or artificial OT were excluded from the study.

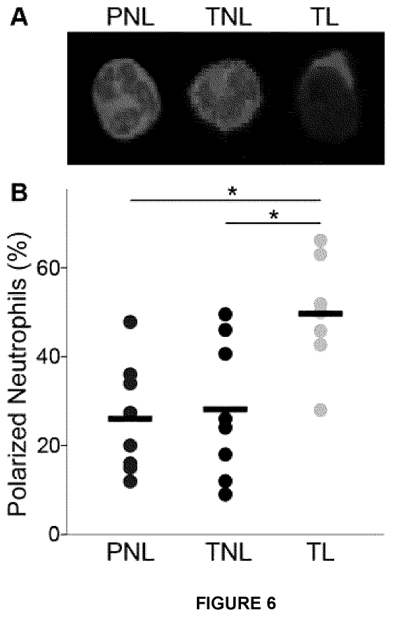

Leukocyte migration assay (LMA). The LMA for FIGS. 6 and 7 was performed as described above with the exception that 200,000 leukocytes were placed into the upper chamber. The LMA for FIGS. 3, 5 and FIG. 10 was performed using non-commercial, 3D printed Boyden chambers that produced nearly identical results to our published work with commercial Boyden chambers with the exception that the cutoff for predicting delivery within 7 days was 4575 cells (see FIG. 10 for more information).

Isolation and Treatment of Fetal Membrane Explants. Intact placentas were collected with consent from TNL women undergoing an elective caesarean section (Royal Alexandra Hospital, Edmonton, AB). Human fetal membrane explants were excised using a 6 mm tissue punch and washed with HBSS. Explants were plated in a 12-well transwell plate with the chorion facing down. Transwells were filled with DMEM F-12 (HyClone, UT, USA) containing 15% fetal bovine serum (FBS) and 1× antibiotic/antimycotic (100 U/mL penicillin G sodium, 100 μg/mL streptomycin sulfate, and 0.25 μg/mL amphotericin B; Hyclone, GE Healthcare Life Sciences. Mississauga, ON, Canada). Explants were acclimatized for 48 h at 37° C. and 5% CO₂. Treatment solutions of 1 ng/mL IL1β (MilliporeSigma, Etobicoke, ON, Canada) in DMEM F-12 were administered over a 6-h incubation period. The conditioned medium was then collected for further processing.

Leukocyte Incubation with hFM Conditioned Medium. Conditioned medium from hFM explants was prepared as described previously (23). TL leukocytes were incubated with this single conditioned medium for 1 h at 37° C. and gentle agitation using a stir bar to prevent clumping. The supernatant was isolated via density centrifugation and used as the chemoattractant in an LMA to assess the further contribution of the leukocytes to the conditioned medium. This is referred to as ‘double conditioned medium.’ The pelleted TL leukocytes were resuspended in RPMI, and their responsiveness to GCF was assessed using an LMA.

Leukocyte Incubation with Maternal Serum. TL or TNL whole blood was left to clot at room temperature for 30 min, after which serum was separated from the whole blood by centrifugation at 1500×g for 10 min at 4° C. The serum was stored at −20° C. Immediately upon collection. 1×10⁷ TNL leukocytes were incubated with the serum at a 50:50 ratio for 1 h at room temperature under gentle agitation using a stir bar. The TL leukocytes were isolated via density centrifugation and resuspended in RPMI. An LMA was used to assess the responsiveness of leukocytes upon stimulation with TL or TNL maternal serum compared to control.

RT-qPCR. RNA was extracted from mouse whole blood using the RNeasy protect animal blood system (Qiagen, Hilden. Germany) according to the manufacturer's protocol, and RNA concentration and integrity was determined using a NanoDrop 1000 spectrophotometer. cDNA was synthesized from 500 ng RNA using iScriptase (Bio-Rad). Primers were designed using the National Center for Biotechnology Information's Primer Blast. These primers are described in Table 1. Quantitative gene analysis was performed using SYBR Green Master Mix (Bio-Rad). Gene expression levels were normalized to β-actin (Actb). Melting curves were used to test primer specificity.

Statistical analysis of incubation experiments. Statistical analysis was performed using Student's t-Test, Pearson's correlation test or two-way ANOVA followed by Tukey's HSD test as a post hoc test. Significance was achieved at p<0.05.

### References

1. Gomez-Lopez N. Guilbert U. Olson D M. Invasion of the leukocytes into the fetal-matemal interface during pregnancy J Leuk Biol 88(4): 625-33, 2010.
2. Timmons B C, Mahendroo M S. Timing of neutrophil activation and expression of proinflammatory markers do not support a role for neutrophils in cervical ripening in the mouse. Biol Reprod 2006; 74: 236-245.
3. Timmons B, Akins M, Mahendroo M. Cervical remodeling during pregnancy and parturition. Trends Endocrinol Metab 2010; 21: 353-361.
4. Thomson A J, Telfer J F, Young A, Campbell S, Stewart C J, Cameron I T, et al. Leukocytes infiltrate the myometium during human parturition: further evidence that labour is an inflammatory process. Hum Reprod. 1999:14(1):229-36.
5. Keski-Nisula L. Aalto M L, Katila M L, Kirkinen P. Intrauterine inflammation at term: a histopathologic study. Hum Pathol 31: 841-6, 2000.
6. Hamilton S. Oomomian Y, Stephen G, Shynlova O. Tower C L, Garrod A et al. Macrophages infiltrate the human and rat decidua during term and preterm labor: evidence that decidual inflammation precedes labor. Biol Reprod 2012; 86: 39.
7. Shynlova O, Nedd-Roderique T, U Y, Dorogin A, Nguyen T, Lye S J. Infiltration of myeloid cells into decidua is a critical early event in the labour cascade and post-partum uterine remodelling. J Cell Mol Med 2013; 17: 311-324.
8. Young A, Thomson A J, Ledingham M, Jordan F, Greer I A, Norman J E: Immunolocalization of proinflammatory cytokines in myometrium, cervix, and fetal membranes during human parturition at term. Biol Reprod 2002, 66(2):445-449.
9. Osman I, Young A, Ledingham M A, Thomson A J, Jordan F, Greer I A, Norman J E: Leukocyte density and pro-inflammatory cytokine expression in human fetal membranes, decidua, cervix and myometrium before and during labour at term. Mol Hum Reprod 2003, 9(1):41-45.
10. Liggins, G. Cervical ripening as an inflammatory reaction. In: Ellwood, DA., Anderson, ABM., editors. The Cervix in Pregnancy and Labour, Clinical and Biochemical Investigation. Churchill Livingston; 1981.
11. Bokstrom H, Brannstrom M, Alexandersson M, Norstrom A: Leukocyte subpopulations in the human uterine cervical stroma at early and term pregnancy. Hum Reprod 1997, 12(3):586-590.
12. Rinaldi S F, Catalano R D, Wade J, Rossi A G, Norman J E. Decidual neutrophil infiltration is not required for preterm birth in a mouse model of infection-induced preterm labor. J Immunol 2014; 192: 2315-2325.
13. Gomez-Lopez N, et al. Immune cells in term and preterm labor. Cellular & Molecular Immunology (2014) 11, 571-581.
14. Robertson S A, et al. Targeting Toll-like receptor-4 to tackle preterm birth and fetal inflammatory injury. Clinical & Translational Immunology 2020; e1121.
15. Gomez-Lopez N, Vadillo-Perez L, Nessim S, Olson D M, Vadillo-Ortega F. Choriodecidua and amnion exhibit selective leukocyte chemotaxis during term human labor. Am J Obstet Gynecol 2011; 204: 5.
16. Gomez-Lopez N, Hernandez-Santiago S. Lobb A P. Olson D M, Vadilo-Ortega F. Normal and premature rupture of fetal membranes at term delvery differ in regional chemotactic activity and related chemokine/cytokine production. Reprod Sci 2013; 20: 276-284.
17. Gomez-Lopez N, Tanaka S, Zaeem Z, Metz G A, Olson D M. Maternal circulating leukocytes display early chemotactic responsiveness during late gestation. BMC Pregnancy Childbirth 2013, 13(Suppl 1): S8.
18. Gomez-Lopez N, Tong W C, Arenas-Hernandez M, Tanaka S, Hajar O, Olson D M, Taggart M J, Mitchell B F. Chemotactic activity of Gestational Tissues Through Late Pregnancy, Term Labor, and RU486-Induced Preterm Labor in Guinea Pigs. Am J Reprod Immunol 73(4): 341-52, 2015.
19. Takeda J, Fang X, Olson D M. Pregnant human peripheral leukocyte migration during several late pregnancy clinical conditions: a cross-section observational study. BMC Pregnancy Childbirth 17:16, 2017.
20. Yuan M, Jordan F, McInnes I B, Hamett M M, Norman J E. Leukocytes are primed in peripheral blood for activation during term and preterm labour. Mol Hum Reprod 2009; 15: 713-724.
21. Christiaens I, Zaragoza D B, Guilbert U, Robertson S A, Mitchell B F, Olson D M. Inflammatory processes in preterm and term parturition. Journal of Reproductive Immunology. Invited review, 79:50-57, 2008.
22. Nadeau-Vallee M, et al. Novel Noncompetitive IL-1 Receptor-Biased Ligand Prevents Infection- and Inflammation-Induced Preterm Birth. Journal of Immunology. 2015 October; 195(7):3402-15. PubMed PMID:26304990, 2015.
23. Leimert K B. et al. Maternal and fetal intrauterine tissue crosstalk promotes pro-inflammatory amplification and uterine transition. Biology of Reproduction 2019 Mar. 1; 100(3):783-797.
24. Fleming, A. F. 1975, Hematological changes in pregnancy. Clin Obstet Gynecol.:2:269.
25. Krause, P J, et al. Host defense during pregnancy: neutrophil chemotaxis and adherence. Am J Obstet Gynecol. 1987:157(2):274-80.
26. Lurie S, et al. Total and differential leukocyte counts percentiles in normal pregnancy. Eur J Obstet Gynecol Reprod Biol. 2008; 136(1):16-19.
27. Karalis L, Nadan S, Yemen E A. 2005, Platelet activation in pregnancy induced hypertension. Thromb Res.; 116(5):377-383.
28. Davis J M. et al., Increased neutrophil mobilization and decreased chemotaxis during cortisol and epinephrine infusions. J Trauma. 1991; 31(6):725-31
29. von Dadelszen P, et al. Maternal neutrophil apoptosis in normal pregnancy, preeclampsia, and normotensive intrauterine growth restriction. Am J Obstet Gynecol. 1999:181(2):408-14.
30. MacCallum D E, et al. Selicidib (CYC202. R-Roscovitine) induces cell death in multiple myeloma cells by inhibition of RNA polymerase II-dependent transcription and down-regulation of Mcl-1. Cancer Res. 2005; 65:5399-5407
31. Berker E, et al. Effect of neutrophil apoptosis on monocytic cytokine response to Porphyromonas gingivalis lipopolysaccharide. Journal of Periodontology 2005; 76:6.
32. Gouwy M. et al. Synergy between proinflammatory ligands of G protein-coupled receptors in neutrophil activation and migration J. Leukoc. Biol. 76: 185-194; 2004.
33. Struyf, S. et al. Chemokines synergize in the recruitment of circulating neutrophils into inflamed tissue Eur. J. Immunol. 2005. 35: 1583-1591.
34. Metzemaekers M, Gouwy M, Proost P. Neutrophil chemoattractant receptors in health and disease: double-edged swords. Cellular & Molecular Immunology (2020) 17:433-450
35. Luppi P. et al. Monocytes are progressively activated in the circulation of pregnant women. J Leukoc Biol. 2002; 72(5):874-884.
36. Naccasha N, et al. Phenotypic and metabolic characteristics of monocytes and granulocytes in normal pregnancy and maternal infection. Am J Obstet Gynecol. 2001:185(5):1118-1123.
37. Luppi P, et al. CD11b Expression on circulating leukocytes increases in preparation for parturition. Am J Reprod Immunol. 2004:52(5):323-329.
38. Gambardella L, Vermeren S. Molecular players in neutrophil chemotaxis-focus on PI3K and small GTPases. J Leuk Biol 2013; 94(4): 603-12.
39. Hirsch E et al. Central role for G protein-coupled phosphoinositide 3-kinase delta in inflammation Science 2000; 287(5455): 1049-53.
40. Sadhu C, et al. Essential role of phosphoinositide 3-kinase delta in neutrophil directional movement. J Immunol 2003: 170(5): 2647-54.
41. Glogauer M, et al. Rac1 deletion in mouse neutrophils has selective effects on neutrophil functions. J Immunol 2003; 170: 5652-57.
42. Weiner O D. et al. A PtdInsP(3)- and Rho GTPase-mediated positive feedback loop regulates neutrophil polarity. Nat Cell Biol 2002; 4: 509-13.
43. Philipson M, et al. Vav1 is essential for mechanotactic crawling and migration of neutrophils out of the inflamed microvasculature. J Immunol 2009; 182(11): 6870-78.

### Example 2

### Abstract

Interleukin (IL)-1β is a mediator of infectious and sterile-inflammatory preterm birth (PTB) that leads to uterine activation and fetal brain disease, accompanied by leukocyte invasion. We developed a mouse Leukocyte Migration Assay (mLMA), and along with rytvela, an IL-1R allosteric antagonist, we studied leukocyte migration, in vitro, to understand better the mechanisms of leukocyte invasion, in vivo. Chemoattractant extracted from mouse lower uterus on gestational day (GD) 18.5 was used to develop the mLMA. The chemoattractant from mice attracted more leukocytes per gram than other mammalian chemoattractants. IL-1 β stimulated PTB at GD 17 and increased neutrophil invasion of the uterus and fetal brain; increased leukocyte mRNA abundance of IL-1 β, IL-6, TNFα and CCL2; and increased migration. Rytvela blocked these effects. IL-1 β did not stimulate uterine chemoattractant production although it stimulated mouse CXCL8 analogs, KC and MIP-2. We conclude that mammals produce chemoattractant in intra-uterine tissues at term that attract leukocytes for labor, this may initiate term parturition. At preterm, IL-1 β stimulates the leukocytes for migration and invasion into the uterus, initiating PTB, and the fetal brain leading to harm. The mLMA is a useful test for assessing inflammatory risk and effectiveness of rytvela treatment.

### Introduction

A conserved phenomenon of term and preterm birth (PTB) among mammalian species is the invasion of circulating leukocytes into the gestational tissues. In humans, leukocyte densities in the fetal membranes, decidua, cervix and myometrium increase significantly at term labor (1, 2). A greater proportion of total innate lymphoid cells infiltrate the decidua parietalis of women in preterm labor (PTL) (3). Moreover, elevated leukocyte counts are found in the amniotic fluid of women who have delivered preterm and exhibited an infectious etiology (4). In mice, both full- and sub-PTB-Inducing doses of the gram-negative bacterial mimic lipopolysaccharide (LPS) stimulate neutrophil infiltration of fetal membranes, and a PTB-inducing dose of LPS induces infiltration of the fetal lung and kidneys (5). Once leukocytes invade, they release an array of matrix metalloproteases, prostaglandins, cytokines, chemokines and other effectors that amplify the inflammatory event, activate the uterus for labor (e.g. altered expression of uterine activation proteins), remodel the cervical extracellular matrix, and break down the fetal membranes (6-8). These events cause parturition.

We and others have studied the mechanisms of leukocyte migration at term and preterm in pregnant women (9-13). The human fetal membranes, amnion and chorion, release a chemoattractant whose levels rise as labor approaches, and the responsiveness of the leukocytes to the chemoattractant also increases as labor approaches. We developed a leukocyte migration assay (LMA) to study this phenomenon in rats, guinea pigs and humans using a Boyden chamber that mimics some of the early in vivo actions of leukocyte invasion (11-13).

Other work by our team has demonstrated clearly that PTB and arrested fetal organogenesis are induced in mice by administration of IL-1β (14-15), a model for sterile inflammation (16), or by lipoteichoic acid, LTA, a gram-positive bacterial mimic and LPS. LTA and LPS interact with the toll-like receptors (TLR) 2 and 4, respectively, which cause PTB by stimulating IL-1β production. This was made evident because PTB was blocked by administration of the IL-1 receptor (IL-1R) antagonist, rytvela, a 7-amino acid allosteric antagonist of the IL-1R. These reports also suggest that IL-1 β is a central mediator of PTB, regardless of whether infection is present.

The purpose of this study was to use these new tools, the LMA and rytvela, to explore the relationship of the migration of human and mouse leukocytes to chemoattractants from various species and the mouse in detail and how IL-1 β affects leukocyte migration in mice and preterm delivery. Our hypothesis was that IL-1 β would stimulate both leukocyte migration and leukocyte invasion at PTB in maternal uterus and fetal brain, and that these effects would be blocked by rytvela.

### Results

IL-1β Induces Neutrophil Invasion of Maternal Uterus and Fetal Brain and PTB Resulting in Fetal Death, but Rytvela Blocks these Actions.

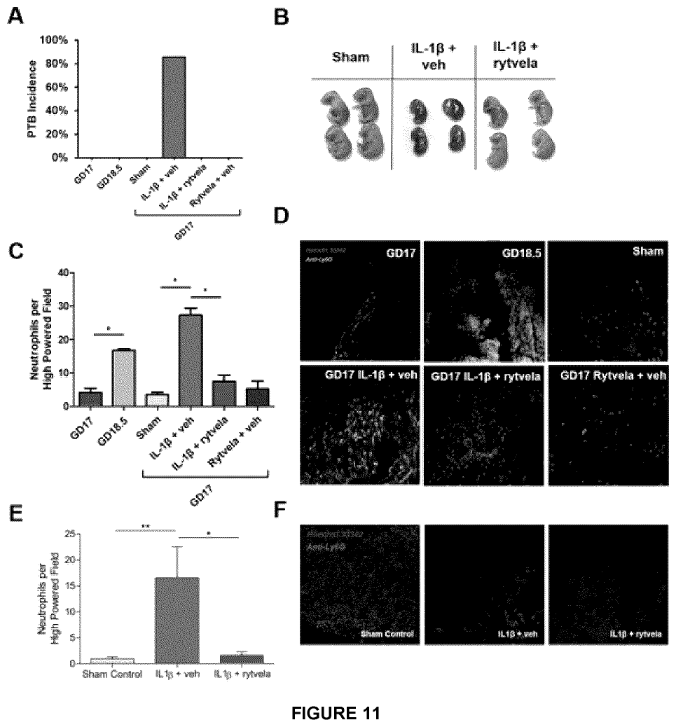

Given the central role that IL-1β plays in mediating both sterile and infectious etiologies of PTB, IL-1β was chosen to induce PTB in our mouse model (14-16). FIG. 11A demonstrates that IL-1β administered on GD 16 stimulated PTB on GD 17 at a rate of 86%, whereas none of the other groups' pregnancies terminated in PTB. This administration of IL-1β led to increased fetal death (FIG. 11B), and increased detection of Ly-6G+ neutrophils in the lower uterus and fetal brain compared to sham control (FIG. 11C-E) which was similar to the increase detected at GD18.5 in untreated mice (p<0.05 compared to GD17 control, FIG. 11C, D). Rytvela co-administration decreased the PTB to 0%, prevented fetal death and blocked the increase in neutrophil density in the maternal uterus and fetal brain (p<0.05. FIG. 11A-E). We explored the basis for these phenomena.

Development of a Mouse Leukocyte Migration Assay (mLMA).

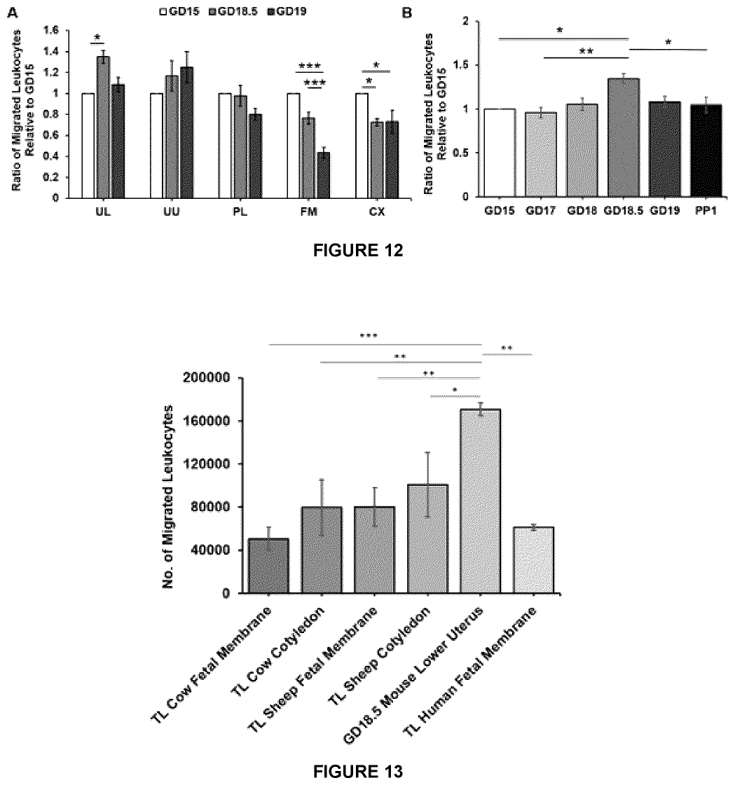

The first step in developing the mLMA was to identify the best source and timing of the chemoattractant. In FIG. 12A we compared lower and upper uterus, placenta, fetal membranes and cervix on GDs 17, 18.5 and 19, which is the day of delivery. We found that the lower uterus produced relatively more chemoattractant, as assessed by human TL leukocyte migration, than the other mouse uterine tissues and that it increased from GD 17 to 18.5, which would correlate with a timing appropriate for initiating labour. These mice delver on GD 19. We then compared extracts of homogenized lower uterine tissue with greater clarity from 4 d before to 1 d after delivery using TL human leukocytes (FIG. 12B). Again, we found a statistically significant increase (p<0.05) in chemoattractant potency at day 18.5, approximately 12 h before delivery, and then a decrease in chemoattractant potency in the lower uterus at 1 d postpartum.

Comparison of Mouse with Other Mammalian Chemotactic Factors

We compared chemotactic factors extracted from human fetal membranes, mouse lower uterus from GD 18.5, bovine fetal membranes, bovine placental cotyledons, ovine fetal membranes and ovine placental cotyledons, al obtained following spontaneous delivery at term, for attraction of human TL leukocytes (FIG. 13). Control chambers had no more than 200 cells migrate (data not shown). The mouse had the highest concentration of chemoattractant on a per gram wet weight basis.

mLMA with Mouse TL Leukocytes

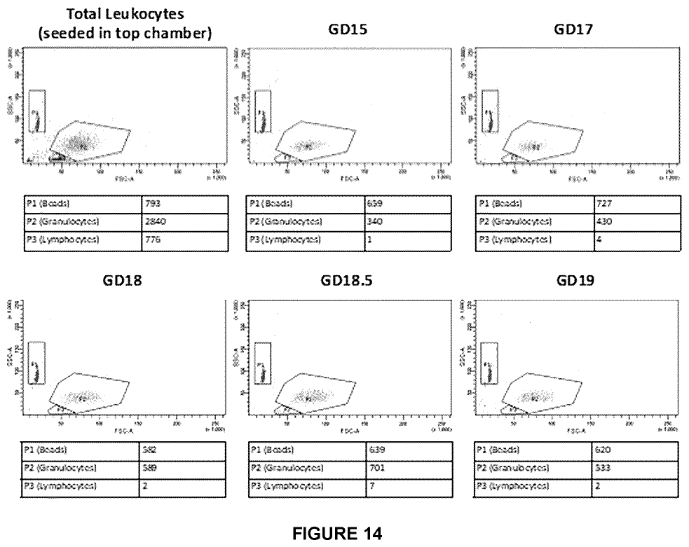

Once the source and timing of chemoattractant was determined, the next step was to use pregnant term mouse leukocytes rather than human TL leukocytes to test their relative attraction to mouse chemoattractant from GD 15 to 19 (FIG. 14). We followed the migration of granulocytes and lymphocytes; the beads demonstrated the flow through the chamber. The flow of control beads was consistent through this four-day period, but there was no attraction of lymphocytes. A marked increase in granulocyte numbers occurred to GD 18.5 and fell on GD 19. These data indicate that both granulocytes and chemoattractant reach their greatest migration attraction on GD 18.5. The implications are that the chemoattractant influences granulocyte activation and migration, and they activate the uterus for delivery.

### Testing for Chemokines and Chemoattractant Production During IL-1β-Induced PTB.

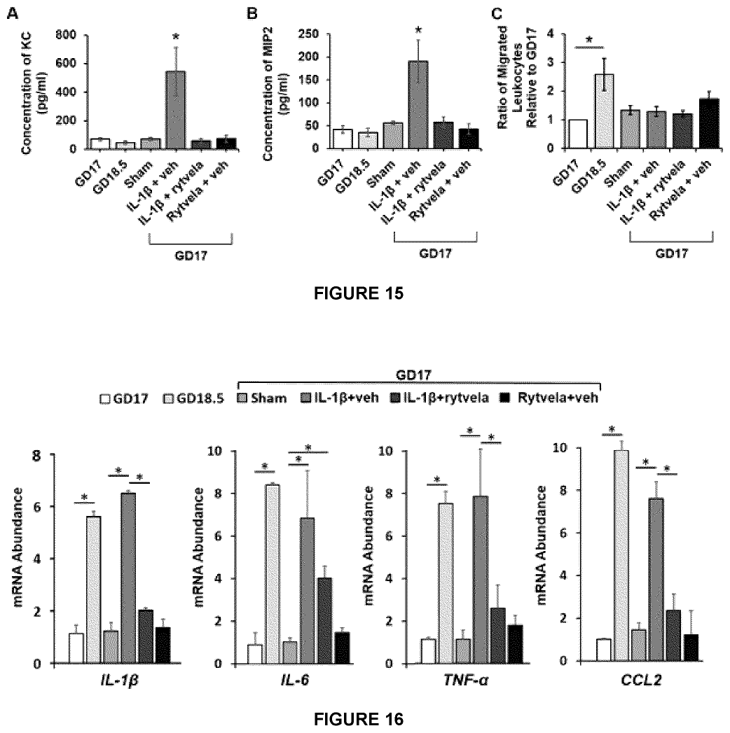

KC and MIP-2 are the functional CXCL8 (also IL-8) homologs in rodents, which do not express the major neutrophil chemoattractant, CXCL8 (17-21). Using multiplex analysis of the lower uterus extracts during normal mouse pregnancy at GD 17 and 18.5 and during IL-1 β-stimulated PTB, we observed that IL-1β stimulated increases in KC and MIP-2 protein but there were no naturally occurring increases at GD 18.5 (FIG. 15A, B). In contrast. IL-1 β did not stimulate an increase in chemoattractant on GD 17, but there was a naturally occurring increase in chemoattractant at GD 18.5 compared to GD 17 We conclude that IL-1 β does not stimulate chemoattractant production to effect PTB. Whether KC or MIP-2 are active constituents in GD18.5 lower uterine chemoattractant remains to be determined.

### IL-1 β Stimulates Leukocyte Activation in the Mouse; Rytvela Blocks This.

Peripheral mouse leukocytes from the experiments in FIG. 11 demonstrated increases in the mRNA abundance of cytokines IL-1 β, IL-6, transforming growth factor (TNF)-α, and the chemokine, CCL2, shortly before normal term delivery on GD 18.5 compared to GD 17 (FIG. 16, p<0.05). A similar increase in mRNA expression of these cytokines was observed on GD 17 following administration of IL-1 β on GD 16 (p<0.05). This was blocked by co-administration of rytvela (p<0.05). These data indicate that leukocyte activation, as indicated by increased cytokine production, did increase at term and in response to IL-1 β treatment.

### IL-1 β Stimulates Increased Pregnant Mouse Leukocyte Migration

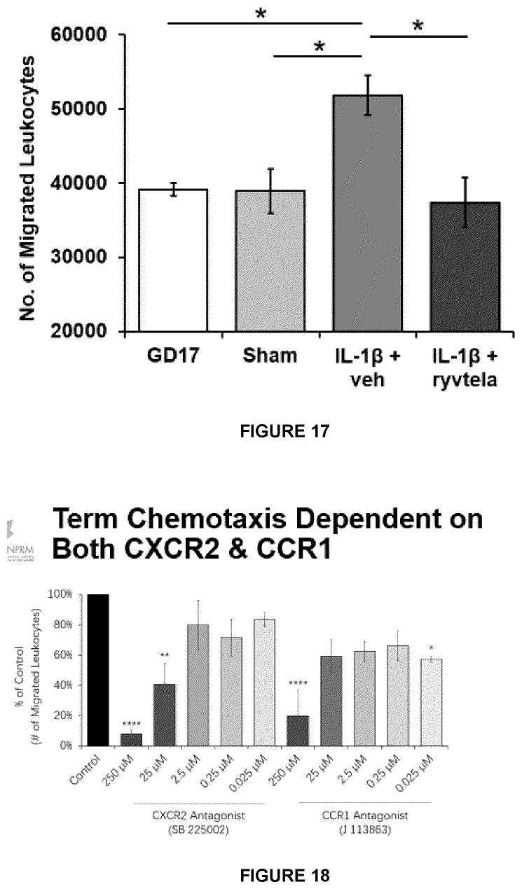

The demonstration that IL-1β stimulates leukocyte activation in terms of cytokine production would suggest it also stimulates increased migration. We observed that administration of IL-1β on GD 16 that produces PTB on GD 17 also increased leukocyte migration on GD 17 (p<0.05). This was blocked by rytvela (FIG. 17).

### Discussion

These data demonstrate in a sterile inflammation murine model of PTB that IL-1β stimulates leukocyte activation as identified by increased cytokine expression, increased leukocyte migration, in vitro, and increased invasion, in vivo, into the uterus and fetal brain. Each of these actions is blocked by co-treatment with rytvela, the small peptide, allosteric IL-1R receptor antagonist. Further we established the mLMA using GD 18.5 lower uterine chemoattractant and demonstrated the highest level of mouse leukocyte migration in response to mouse chemoattractant was GD 18.5, about 12 h prior to delivery (FIG. 14). It is possible that uterine chemoattractant increases leukocyte activation and migratory response to it. The chemoattractants of gestational tissues (placenta, fetal membranes or uterus) from many species are likely chemically similar since they al attract TL human leukocytes. TL human leukocytes respond to mouse gestational chemoattractant in a tissue-specific and gestational age-dependent manner with the greatest migratory response to chemoattractant from the lower uterus at GD 18.5. This coincides precisely with actual neutrophil invasion in terms of uterine location and time of gestation (FIG. 11). The action of IL-1β is most likely at the level of leukocyte activation and invasion into tissues to activate the uterus to effect labor as opposed to causing a direct increase in uterine chemoattractant. At term delivery, the data strongly suggest that fetal signals increase chemoattractant production (and possibly its composition) naturally in late pregnancy to signal the timing of delivery.

Our study adds to the existing literature on gestational chemoattractant, which has been identified previously in rats, guinea pigs and humans (9-13). We showed that chemoattractants isolated from term cow and sheep cotyledons and several intrauterine tissues in the mouse during late pregnancy were able to attract human TL leukocytes (FIG. 13). These data suggest that release of chemokines from several species and their abilities to attract the leukocytes of term labor pregnant women speaks to a fundamentally important and universal mechanism of naturally terminating pregnancy. It is most likely that these signals are generated by the fetus, and not from IL-1β stimulation (FIG. 15C), since several originate from the placenta (cow, sheep, mouse) or fetal membranes (human) and they increase dramatically shortly before delivery (FIG. 12,13) (9,13).

In other, unpublished data, our work indicates that conditioned medium from term human fetal membranes (similar to our extracted chemoattractant; cf. FIG. 13) activates peripheral vein human term leukocytes to increase their migratory responsiveness to TL human fetal membrane chemoattractant and to release their own chemoattractant. The timing of the increased level of mouse chemoattractant and increased migration of mouse leukocytes both occurring at GD 18.5 would suggest a similar process in the mouse. That is prior to normal onset of labour, the lower uterine chemoattractant contributes to activating peripheral leukocytes to become more sensitive to chemoattractant for migration and then for releasing their products once they migrate to the uterus.

Leukocytes, especially granulocytes plus monocytes and macrophages, are activated in late gestation to invade the uterus to release their products in order to further the transition of the uterus from a pregnant to a parturient organ (6, 22-24). Shynlova et al. observed a significant increase in neutrophil and macrophage numbers in the mouse decidua on GD 18 which rose to reach a peak at 2-6 h postpartum (25). They also showed increased mRNA and protein levels of cytokines and chemokines that were elevated on GD 18 but peaked at term labour and postpartum. Similar results were observed by Edey et al. who demonstrated an increase in neutrophils and monocytes in the pregnant mouse uterus on GD 18 with a fall at parturition, but the highest levels of cytokine and chemokine mRNA and protein levels were at term parturition on GD 19 (26). Overall, the timing of events as described by these studies coincides extremely closely with our demonstrated increases in the abundance of lower uterus chemoattractant (FIG. 12), increased neutrophils in the lower uterus (FIG. 11), increased migration of neutrophils (FIG. 14) and increase of cytokine and chemokine mRNA on GD 18.5 (FIG. 16). Together these actions contribute to promoting the parturient state of the uterus through stimulation of prostaglandins and their receptors, stimulation of more cytokines, and recruitment of more leukocytes. They also contribute to uterine involution that follows delivery of the fetuses and other products of conception (22-27).

The role of IL-1 β at preterm in the mouse is very clear; it stimulates leukocyte activation in terms of cytokine and chemokine release (FIG. 15) (14), neutrophil invasion of the uterus and of the fetal brain (FIG. 11) (15) and increased migration in general (FIG. 17). It does not cause an increase in lower uterine chemoattractant even though it does stimulate two mouse analogs of CXCL8, KC and MIP-2 The available evidence suggests that the role of IL-1 β in leukocyte invasion is to enhance the ability of circulating leukocytes to migrate and invade. Considering that IL-1 β is a central mediator of both infectious and sterile-inflammatory models of PTB, our work contributes to the existing work that draws an association between PTB and leukocyte invasion (3-5, 27). We propose that IL-1β enhances the ability of circulating leukocytes to migrate and invade the uterus. The question derives that perhaps the chemoattractant necessary for leukocyte invasion of the maternal uterus at IL-1 β-induced PTB is produced by leukocytes and not the uterus as it is at term delivery. Future work will explore this possibility.

Equally intriguing is how IL-1 β entices fetal leukocytes to invade into the fetal brain. Ly6G+ neutrophils have been detected in both fetal lung and kidney tissue following maternal administration of sub-labor-inducing LPS doses (5). While it is likely that neutrophils invaded other fetal tissues which we did not examine, to invade the brain they would have had to overcome the blood-brain barrier. In the presence of a severe fetal inflammatory insult, this barrier is compromised (28). In this and previous work, IL-1 β was administered into the intrauterine space between two adjacent amnionic sacs (14, 15). While it is known that cytokines can diffuse between cells (29, 30), it has not been demonstrated that cytokines diffuse from mother to the fetus. The available data would suggest that they propagate from mother to fetus via molecular and cellular pathways that include stimulation of their own synthesis in a cell-to-cell fashion (7, 8, 31). The same question applies to rytvela which was administered subcutaneously. Our evidence indicates that it does not transfer from mother to fetus in the mouse (15). This information suggests that rytvela blocks IL-1 β action systemically in the dam which then stops IL-1β from a cell-to-cell stimulation of its own synthesis across the placenta or fetal membranes to the fetus. These are interesting questions for future experimentation, and they derive whether elevated IL-1 β in the fetus stimulates activation of the fetal neutrophils such that they then invade the fetal brain.

One of the potential translation implications of this work is summarized by combining the information from FIGS. 11 and 17 whereby the mLMA is able to indicate both when the height of inflammatory invasion by leukocytes occurs in the maternal uterus and fetal brain and when rytvela blocks these actions and restores normality in terms of leukocyte migration and health. These data demonstrate that the LMA can be used to inform when inflammation and leukocyte migration are high and when rytvela treatment is successful. Without such a test, there would be no other indicators in cases of subclinical infection to know when treatment is successful. Thus, the LMA can be a proxy for assessing fetal inflammation and rytvela treatment success.

### Materials and Methods

### Mice

CD-1 mice were ordered from Charles-River Laboratories Canada (Montreal, QC, Canada) and housed by Health Sciences Laboratory Animal Services at the University of Alberta. (Edmonton, AB, Canada) on a standard diet. Six groups of mice were euthanized at GD15, 17, 18, 18.5, 19 during spontaneous labor, and post-partum (PP)-1 Four groups of mice were anesthetized under isoflurane and a 1.5 cm-tall median incision was made in the lower abdominal wall. The lower segment of the right uterine hom was exposed, IL-1β (3 μg) or vehicle (0.9% saline) was injected between two fetal membranes, and the abdominal muscle layer and skin were sutured. Rytvela (1 mgikg, Elim Biopharmaceuticals, Hayward, CA) or vehicle (0.9% saline) was injected i.p. in a total volume of 100 μL saline, the first dose of which was given 30 min prior to administration of stimuli and three doses every 12 h thereafter. Animals were monitored for the onset of preterm labor (PTL) using infrared video camera recording commencing at GD16.5. Mice were euthanized at the onset of PTL or at GD17 From all ten groups of mice, lower uteri, upper uteri, placentas, fetal membranes and cervices were collected and normalized to wet tissue weight (100 mg/mL) in Dulbecco's Modified Eagle's Medium (DMEM) F-12 (Thermo Fisher Scientific, MA, USA). These tissues were homogenized by TissueLyser II (QIAGEN, Hilden, Germany) according to the manufacturer's protocol and centrifuged at 4° C., 13,000×g for 10 min to extract total protein. Maternal whole blood was collected via cardiac puncture. Whole fetuses (n=2 per dam) were also collected.

### Other Tissues

Cow (n=3) and sheep (n=3) placental tissues were generous gifts from the Gubbels laboratory (Augustana University. Sioux Falls, SD, USA). Intact tissues were collected following delivery and washed thoroughly with PBS. Whole fetal membranes were excised using a 6 mm tissue punch. Approximately 8 mm3 cubic sections of whole cotyledons (both maternal and fetal halves) were excised using scissors. Whole human placentas (n=6) were collected from term labor (TL) pregnant women at the Royal Alexandra Hospital (Edmonton, AB, Canada) and washed thoroughly with PBS, and fetal membranes were excised using a 6 mm tissue punch. All tissues were normalized to wet tissue weight in DMEM F-12 (100 mg/mL), homogenized by TissueLyser II and centrifuged at 4° C., 13,000×g for 10 min to extract total protein.

### Human Leukocytes

Human whole blood was collected following informed consent from TL pregnant women at the Royal Alexandra Hospital (Edmonton, AB, Canada). Labor was documented by a cervical dilation of greater or equal to 4 cm in the presence of uterine contractions. Women with clinical infection, premature rupture of membranes, diabetes mellitus, immunological problems, non-singleton pregnancies, intrauterine growth restriction, preeclampsia or dysfunctional labor, and recipients of progesterone or oxytocin were excluded from the study.

### Mouse Leukocytes

Mouse whole blood (1-2 mL) was obtained by cardiac puncture and its leukocytes were processed for the LMA similarly to human leukocytes (below).

### Leukocyte Migration Assay (LMA)

Total leukocytes were isolated from whole blood using HetaSep (Stemcell Technologies Canada Inc., BC. Canada) according to the manufacturer's protocol. Leukocytes were resuspended in Roswell Park Memorial Institute medium 1640 (RPMI 1640) and diluted to 200,000 cells per 100 μL. Chemoattractants (50 μL) were loaded into the lower wells of a 96-well chemotaxis chamber (Neuro Probe Inc., MD, USA) and a polycarbonate filter (3 μm pores) was placed on top. Total leukocytes (200.000 cells in 100 μL) were loaded into the upper wells and were left to migrate towards the chemoattractant at 37° C. in 5% CO2 for 30 min. Chemoattracted leukocytes were transferred to a 96-well, black, clear bottom plate. Measurements were taken in triplicate determination. Hoechst 33342 (1 pM) was added to reach a final volume of 100 μL per well. Fluorescence in each well was measured using a Fluoroskan Ascent (Thermo Fisher Scientific), and the number of chemoattracted leukocytes was calculated using 4 parameter logistic regression.

### Immunohistochemistry

Six cryosections (7 μm) per treatment group were prepared from snap-frozen lower uteri and fetal heads (n=2 per dam) and stained for neutrophils using a goat anti-mouse Ly-6G antibody (Invitrogen, Carlsbad, CA, USA) and a rat anti-goat secondary antibody tagged with an Alexa Fluor 488 fluorescence marker (Invitrogen). Cells were counterstained using Hoechst 33342 (Thermo Fisher Scientific) and tissue homology was confirmed with a hematoxylin and eosin stain (Thermo Fisher Scientific). Four different fields (20× optical zoom) were counted per section by two observers blinded to the specimen details. Areas containing blood vessels and leukocytes within blood vessels were excluded. The arithmetic mean was calculated for each sample between the two observers. In the fetus, we focused on leukocyte invasion of the brain because of the evidence associating maternal inflammation and adverse outcomes to the fetal brain (15, 28).

### RNA Extraction and RT-qPCR

Total RNA was extracted from mouse whole blood using the RNeasy protect animal blood system (Qiagen, Hilden, Germany) according to the manufacturer's protocol. RNA concentration were determined using a NanoDrop 1000 spectrophotometer to measure the optical density (OD) photometrically at 280 nm and 260 nm (an OD260 nm/280 nm ratio of >1.8 was considered protein-free RNA). cDNA was synthesized from 500 ng RNA using qScript cDNA SuperMix (Quanta Biosciences, MA, USA) according to the manufacturer's protocol. Primers for mouse Il-1β, Il-6, Tnf-α, and Ccl2 were designed using the National Center for Biotechnology Information's Primer Blast. Primer sequences are described in Table 1. An annealing temperature of 60° C. was used for all primers. Quantitative gene expression analysis was performed on iCycler IQ (Bio-Rad, CA, USA) using SYBR Green Master Mix (Bio-Rad) according to the manufacturer's protocol. Melt curve analysis was performed to ensure that the amplification of non-specific products did not occur. Standard curves for both the target genes and cyclophilin A (CypA) were generated by serial dilutions of the cDNA samples. Target gene levels were expressed relative to CypA.

### Chemokine Extraction and Mass Estimation

Mouse lower uterus tissue was suspended in DMEM-F12 medium (Thermo Fisher Scientific Inc., Ottawa, ON, Canada) at 100 mg wet tissue weight/mL and homogenized by TissueLyser II (Qiagen, Hilden, Germany) according to the manufacturer's instructions. The lysate was centrifuged at 13,000×g in 4° C. for 10 min to isolate total protein. This protein extract was diluted 1:4 prior to use in the LMA. Keratinocyte chemoattractant (KC) and macrophage inflammatory protein (MIP)-2 levels were measured by Bio-Plex Multiplex Immunoassay kit (Bio-Rad Laboratories, Hercules, CA, USA) according to the manufacturer's instructions.

### Statistics

The data were tested by the D'Agostino-Pearson normality test for normality using GraphPad Prism 8.0. Data that did not pass the normality test were log-transformed to achieve normal distribution. The statistical significance was tested by one-way ANOVA. When a significant F value was produced, means were differentiated using Tukey's post hoc multiple comparisons test. Significance was achieved at p≤0.05.

### Study Approval

Animal studies at the University of Alberta were conducted in accordance with the Canadian Council on Animal Care Guidelines and Policies with approval from the Animal Care and Use Committee: (Biosciences, Health Sciences or Livestock) for the University of Alberta. Studies at Augustana University were performed in accordance with and the approval of the Institutional Research Ethics Board.

Human leukocytes and fetal membranes were obtained following informed consent: the University of Alberta/Alberta Health Services Institutional Review Board approved the study.

TABLE 1
RT-qPCR primers for mouse cytokines

| | Forward Primer | Reverse Primer |
|---|---|---|
| IL1β | 5′-AGATGAAGGGCTGCTTCCAAA-3′ | 5′-GGAAGGTCCACGGGAAAGAC-3′ |
| | (SEQ ID NO: 1) | (SEQ ID NO: 2) |
| IL6 | 5′-CAACGATGATGCACTTGCAGA-3′ | 5′-TCTCTCTGAAGGACTCTGGCT-3′ |
| | (SEQ ID NO: 3) | (SEQ ID NO: 4) |
| TNFα | 5′-GCCTCTTCTCATTCCTGCTTG-3′ | 5′-CTGATGAGAGGGAGGCCATT-3′ |
| | (SEQ ID NO: 5) | (SEQ ID NO: 6) |
| CCL2 | 5′-GCTCAGCCAGATGCAGTTA-3' | 5′-TGTCTGGACCCATTCCTTCT-3′ |
| | (SEQ ID NO: 7) | (SEQ ID NO: 8) |

### References

1. Osman 1, et al. Leukocyte density and pro-inflammatory cytokine expression in human fetal membranes, decidua, cervix and myometrium before and during labour at term. Mol Hum Reprod 2003; 9:41-5.
2. Thomson A J, et al. Leukocytes infiltrate the myometrium during human parturition: further evidence that labour is an inflammatory process. Hum Reprod 1999; 14:229-36.
3. Xu Y, et al. Innate lymphoid cells at the human maternal-fetal interface in spontaneous preterm labor. Am J Reprod Immunol 2018; 79:e12820.
4. Romero R, et al. Amniotic fluid white blood cell count: a rapid and simple test to diagnose microbial invasion of the amniotic cavity and predict preterm delivery. Am J Obstet Gynecol 1991; 165:821-30.
5. Hudalla H. et al. LPS-induced maternal inflammation promotes fetal leukocyte recruitment and prenatal organ infiltration in mice. Pediatr Res 2018; 84:757-64.
6. Orsi N M. Tribe R M. Cytokine networks and the regulation of uterine function in pregnancy and parturition. J Neuroendocrinol 2008; 20:462-9.
7. Leimert K B, et al. Maternal and fetal intrauterine tissue crosstalk promotes proinflammatory amplification and uterine transistion. Biol Reprod 2019 Mar. 1; 100(3):783-797.
8. Leimert K B. et al. Cooperative effects of sequential PGF2a and IL-Ib on IL-6 and COX-2 expression in human myometrial cells. Biol Reprod 2019 May 1; 100(5):1370-1385.
9. Gomez-Lopez N, et al. Fetal membranes exhibit selective leukocyte chemotaxic activity during human labor. J Reprod Immunol 2009; 80:122-31.
10. Yuan M, et al. Leukocytes are primed in peripheral blood for activation during term and preterm labour. Mol Hum Reprod 2009; 15:713-24.
11. Gomez-Lopez N. et al. Maternal circulating leukocytes display early chemotactic responsiveness during late gestation BMC Pregnancy Childbirth 2013:13 Suppl 1:S8.
12. Gomez-Lopez N. et al. Chemotactic activity of gestational tissues through late pregnancy, term labor, and RU486-induced preterm labor in Guinea pigs. Am J Reprod Immunol 2015; 73:341-52.
13. Takeda J, Fang X, Olson D M. Pregnant human peripheral leukocyte migration during several late pregnancy clinical conditions: a cross-sectional observational study. BMC Pregnancy Childbirth 2017; 17:16.
14. Nadeau-Vallée M, et al. Novel Noncompetitive IL-1 Receptor-Biased Ligand Prevents Infection- and Inflammation-Induced Preterm Birth. J Immunol 2015; 195:3402-15.
15. Nadeau-Vallée M. et al. Antenatal Suppression of IL-1 Protects against Inflammation-Induced Fetal Injury and Improves Neonatal and Developmental Outcomes in Mice. J Immunol 2017; 198:2047-62.
16. Romero R, et al. Prevalence and clinical significance of sterile intra-amniotic inflammation in patients with preterm labor and intact membranes. Am J Reprod Immunol 2014; 72(5):458-474.
17. Singer M, Sansonetti P J. IL-8 is a key chemokine regulating neutrophil recruitment in a new mouse model of Shigella-induced colitis. J Immunol 2004:173(6): 4197-206.
18. Sekido N et al. Prevention of lung reperfusion injury in rabbits by amonoclonal antibody against interleukin-8. Nature 1993; 365:654-657.
19. Belperio J A, et al. CXCR2/CXCR2 ligand biology during lung transplant ischemia-reperfusion injury. J Immunol 2005; 175:6931-6939.
20. Souza D G, et al. Repertaxin, a novel inhibitor of rat CXCR2 function, Inhibits inflammatory responses that follow intestinal ischemia and reperfusion injury. Br J Pharmacol 2004; 143:132-142.
21. Miura M, et al. Neutralization of Gro a and macrophage inflammatory protein-2 attenuates renal ischemia/reperfusion injury. Am J Pathol 2001; 159:2137-2145.
22. Robertson S A, et al. The role of cytokines in gestation. Crit Rev Immunol 1994; 14:239-292.
23. Gomez-Lopez N, Guilbert L J, Olson D M. Invasion of the leukocytes into the fetal-maternal interface during pregnancy. J Leukoc Biol 2010; 88:625-633.
24. Gomez-Lopez N. et al. Immune cells in term and preterm labor. Cell Mol Immunol 2014; 11:571-581.
25. Shynlova O, et al. Infiltration of myeloid cells into decidua is a critical early event in the labour cascade and post-partum uterine remodelling. J Cell Mol Med 2013; 17:311-324.
26. Edey L F, et al. Progesterone, the maternal immune system and the onset of parturition in the mouse. Biol Reprod 2018; 98(3):376-395.
27. Shynlova O, Nedd-Roderique T, Li Y, Dorogin A. Lye S J. Myometrial immune cells contribute to term parturition, preterm labour and post-partum involution in mice. J Cell Mol Med 17(1): 90-102, 2013.
28. Paton M C B, et al. Perinatal brain injury as a consequence of preterm birth and intrauterine inflammation: designing targeted stem cell therapies. Front Neurosci 2017; 11:200.
29. Vitkovic L. et al. Cytokine signals propagate through the brain. Mol Psychiatry 2000; 5:604-615.
30. Oyler-Yaniv A, et al. A tunable diffusion-consumption mechanism of cytokine propagation enables plasticity in cell-to-cell communication in the immune system. Immunity 2017; 46:609-620.
31. Ishiguru T, at al. Interleukin (IL)-1 in rat parturition: IL-1 receptors and 1 and 2 and accessory proteins abundance in pregnant rat uterus at term—regulation by progesterone. Physiol Reports 2016; 4(14):e12866.

### Example 3

### Background

The LMA is a functional test of the migration of leukocytes (obtained from a blood sample from an arm vein of a pregnant woman) towards chemoattractants extracted from fetal membranes (amnion and chorion) obtained following delivery. Extensive changes in the methodology making the test much less expensive and much less time-consuming, and validation in a non-Canadian population.

One of the key mechanisms that are involved in the recruitment of these leukocytes to the gestational tissues is the enhancement of leukocytes for chemotaxis towards gestational chemoattractants, a concept that may also be refer to as enhanced leukocyte chemotaxis. Human leukocytes collected at term and preterm are more responsive to human fetal membrane chemoattractants than their non-laboring counterparts

The LMA measures enhanced leukocyte chemotaxis in vitro by the migration of leukocytes across a filter with 3 μm pores in a Boyden Chamber. This is a smaller diameter than that of the leukocytes, requiring them to undergo an active process to squeeze through the pore and move in a non-random direction toward the chemotactic signal.

FIG. 18 depicts a graph depicting term chemotaxis dependent on both CXCR2 and CCR1.

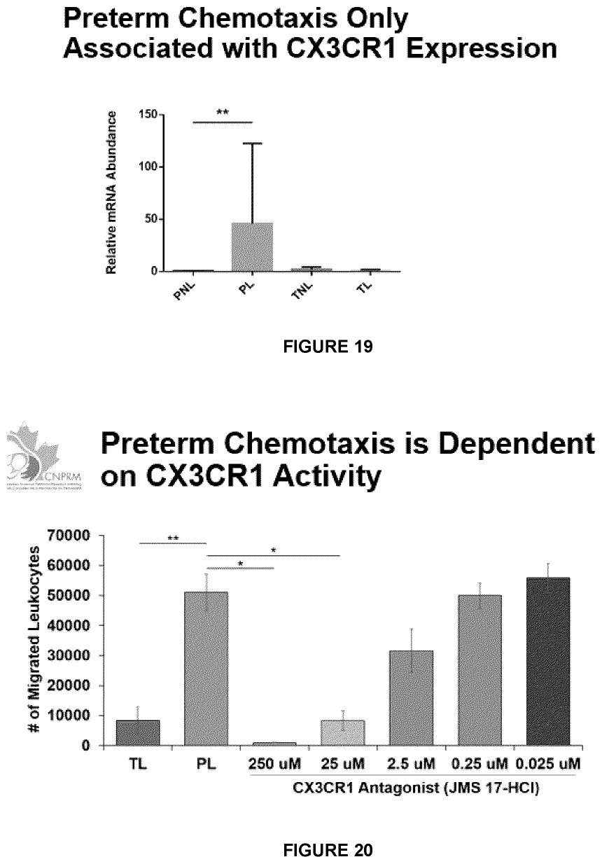

FIG. 19 depicts preterm labor chemotaxis only is associated with CX3CR1 expression.

FIG. 20 depicts preterm chemotaxis is dependent on CX3CR1 activity.

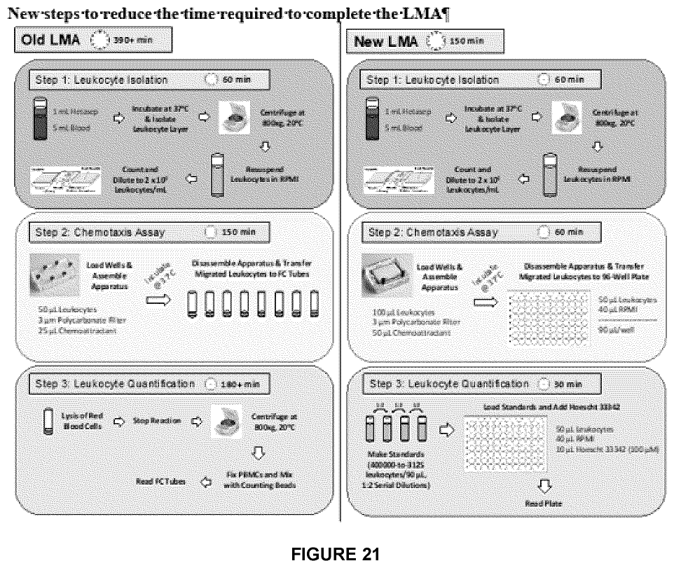

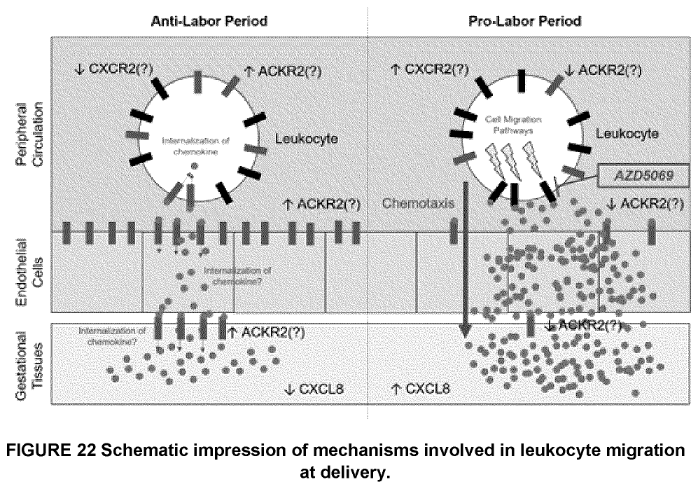

FIG. 21 depicts the time required to complete the LMA.

### Methodology

One example of the method being employed is described herein can be broken down into three basic steps: chemoattractant preparation, leukocyte isolation and chemotaxis assay.

Chemoattractant Preparation. Whole placentas from term human vaginal deliveries without complications are collected following consent from women. They are transported on ice to the diagnostic facility where they are washed thoroughly in phosphate-buffered saline. The fetal membranes (chorion, amnion) are separated from whole placentas using surgical forceps, normalized to wet tissue weight (25 mg/mL) in Dulbecco's Modified Eagle's Medium F12 (Thermo Fisher Scientific, MA, USA), homogenized by TissueLyser II (QIAGEN, Hilden, Germany) at a frequency of 25/s for 6 min and centrifuged at 4 degrees celsius, 13,000×g for 10 min to extract total protein. LMA chemoattractants will be prepared by pooling fetal membrane protein extracts, which will be tested using control leukocytes prior to aliquoting and storage in −80 degrees celsius.

Leukocyte Isolation: Whole blood is collected from a pregnant woman into an EDTA-coated collection tube and incubated for 15 min at room temperature with HetaSep (Stemcell Technologies Canada Inc., BC, Canada), an erythrocyte sedimentation reagent. The total leukocyte fraction is washed in phosphate-buffered saline, centrifuged at 3,000×g for 10 min at room temperature and resuspended in Roswell Park Memorial Institute medium 1640. The concentration of total leukocytes in this solution is counted using a hemocytometer and the solution is diluted to 2,000.000 cells/mL.

Chemotaxis Assay: Chemoattractants (50 μL) are loaded into the lower wels of a simple, resin 3D printed, 3-well chemotaxis chamber and a polycarbonate filter with 3 μm pores (Neuro Probe Inc., MD, USA) is placed on top of the wells. A silicone gasket with holes for each well is positioned on top of the filter and the upper chamber is lowered onto the silicone gasket, creating a tight seal. Total leukocytes (100 μL) are loaded into the wells of the upper chamber and the apparatus is incubated at 90 min at 37 C in 5% CO₂. The apparatus is then disassembled and leukocytes that have migrated through the filter into the lower chamber are quantified using a hemocytometer, flow cytometry or fluorescence spectrometry.

### Performance

Table 1 in s FIG. 10D lists the original performance of the assay in Edmonton using the original method and a validation assay using the new, modified version of the assay methodology and performed by a different operator. The characteristics of each test were nearly identical and validate the new quicker and cheaper methodology and the universality of the assay. In each case the positive predictive value is 91%, the negative predictive value varies between 74-78%, and the area under the curve (AUC in graph) is 83% with high sensitivity and specificity. Also, in each case the assay predicts delivery within 7 days. This is of value in several situations, for instance when women are more than 40 weeks pregnant but not showing clinical signs of labour and contemplating a labour induction. Or when assessing risk of delivery in women who are <37 weeks of gestation and showing at least one symptom of early labour, such as uterine contractions, ruptured membranes or dilated (or short) cervix. In these cases, 85% of women will still deliver at term. In many countries the question becomes whether these women should be admitted to hospital or sent home. With a high positive and negative predictive value, the LMA can be used to discriminate between these two choices.

Predicting the timing of labour onset or delivery is an extremely large international unmet medical need, and at present there are no effective diagnostics on the market to meet the demand for products.

There is an extremely large need for a test that can accurately predict the onset of labour and delivery in a few days for women at term or preterm. These various clinical situations include delivery at term in asymptomatic women who might have gestations lasting longer than 40 weeks, timing of labour onset in women who will have scheduled cesarean sections (about 28% of all deliveries at term) for a variety of clinical conditions (it is preferable to not perform a cesarean section in a laboring woman), preterm delivery in asymptomatic women, preterm delivery in women with ruptured membranes, or risk of early delivery (or not) in women with clinical symptoms of labour. In this latter case, 85% of women with preterm clinical signs of labour will have the symptoms resolve and then deliver at term. The LMA is capable of discriminating between those who will go into labour in 7 days and those who will not with a high level of precision. It is believed that having a more accurate estimation of a mother's due date can help reduce a woman's anxiety about the onset of labor and allow her to make appropriate plans to spend time in hospital.

In addition new animal data suggests that the LMA is an effective test for assessing or confirming a subclinical or clinical infectious process in mothers that may have harmful effects upon the fetus. Inflammation from mother can cross the placenta to the fetus and initiate an inflammatory reaction in the fetus. Data demonstrate that in these cases there can be an arrest of maturation and development of several organs including the brain, lungs and intestine. We have determined that the inflammatory process in the mother stimulates an increase in leukocyte migration that the LMA readily detects that precisely correlates with fetal inflammation. Further, treatment of dams with anti-inflammatory drugs that reduce the maternal and fetal inflammation and restore normal fetal organ development also causes a decrease in the migration of the leukocytes that the LMA also detects. In essence, in some aspects, the LMA can detect maternal and fetal inflammation in asymptomatic pregnant mothers and then become a diagnostic tool to indicate when anti-inflammatory treatment is effective. The annual incidence of preterm birth worldwide is approximately 15 million, and acute complications associated with preterm birth are the leading cause of death in children under the age of 5. In 2012, it was estimated that China contributed to 7.8% of total premature births globally, making them second in the world only to India. This number is likely an underestimate because births at 20-28 weeks were not recorded in accordance with the tenth revision of the International Classification of Disease (ICD-10). In the U.S., the preterm birth rate is nearly 500,000 per year. The estimated societal economic burden associated with preterm birth in the U.S. is estimated to be over $26.2 billion, or approximately $32.325 USD per preterm infant, about 10 times greater than a term infant. Moreover those who survive prematurity are at greater risk of disease and lifelong disabilities. There is a clear need for a better way to assess the risk of preterm birth. Knowing who to treat and who not to treat would be a great benefit to care providers, reduce unnecessary risk for patients and their babies, and reduce healthcare costs by guiding medical decisions regarding the effective use of tocolytic, antibiotic, anti-inflammatory and corticosteroid therapies, and also whether or not transfer to a tertiary care facility is required.

Currently the decision to intervene or not in preterm women is based largely on risk factors, and these are very poor indicators. For instance, a previous preterm delivery increases the risk to about 15% In the current pregnancy, but this is not much higher than the global average risk of 10%. The etiology of preterm labour is multifactorial. Maternal risk factors include maternal age, previous history of preterm delivery, multiple gestation, reproductive tract abnormalities and infection. In women who are asymptomatic, previous history of preterm birth remains the strongest predictor of preterm birth.

Clinical diagnosis of preterm labor is based on symptoms which include frequent uterine contractions in the presence of cervical changes (dilation and effacement) before 37 weeks gestation. Women who present with an initial cervical dilation of 23 cm and at least 80% cervical effacement are assigned a diagnosis of PTL and aggressively treated to delay delivery, if possible, or prepared for delivery. However, if the physical examination does not immediately confirm a diagnosis of progressive PTL, the symptomatic woman is hospitalized for an initial period of observation to determine if the symptoms will subside or progress. Ultrasound assessment of cervical length has been shown to be predictive for symptomatic women, with a short cervix at the second trimester assessment associated with a higher risk for preterm birth.

However, a clinical diagnosis is often unreliable and results in over-diagnosis of PTL. In a Phase III clinical study of Atosiban for treatment of preterm labour, only approximately 50% of untreated patients meeting all entry criteria for diagnosis of preterm labour delivered within 7 days (Romero et al, 2000). The 2008 IHE report states that as few as 1 in 20 PTL cases result in PTD within the next 14 days and that false positive diagnoses on strictly clinical criteria run as high as 50% and true PTL may be missed in 15% to 20% of cases. Therefore a test with both high positive and negative predictive values is necessary to discriminate in these cases.

Existing biomarker-based diagnostics for PTB include the Fetal Fibronectin test by Hologic and the PAMG-1 test by QIAGEN. The former assesses the concentration of fetal fibronectin obtained from a cervical swab. The sample (which can be frozen if needed) is analyzed by a lateral flow, solid-phase immune-chromatographic assay. This is provided by Hologics as a single-use disposable cassette. After 20 min of reaction time, intensities of the test and control lines are interpreted with the company's proprietary TLiIQ Analyzer which must be purchased by the laboratory. One disadvantage of this test is that it cannot be performed within 24 hours of a cervical exam, transvaginal ultrasound or sexual intercourse as this will increase the yield of a false positive result. Another is that the low positive predictive value (around 2% vs. a negative predictive value of 98%) limits its use as a test to predict when delivery will occur. Alberta Health Services has delisted (will not pay for the test) use of this test due to the lack of evidence that it saves costs.

The PAMG-1 test assesses the concentration of placental alpha microglobulin-1 (PAMG-1) from a cervical swab. Similar to Hologic's fetal fibronectin test in that the sample is analyzed by a lateral flow, sold-phase immune-chromatographic assay, which can be visualized by the appearance of one or two lines indicating a ‘no’ and “yes” result respectively. Unlike the fetal fibronectin test, this analysis does not require a laboratory analyzer as it provides an immediate readout, similar to a pregnancy test, and does not require any special equipment. As well, it has a higher positive predictive value than the fetal fibronectin test.

A limitation of both the fetal fibronectin and placental alpha microglobulin-1 tests are that they have only been approved by the U.S. Food and Drug Administration for use in symptomatic preterm women, because they suffer from a poor positive predictive value in asymptomatic women. This fact prevents these diagnostics from aiding physicians to treat the fetal inflammation associated with preterm birth. The LMA overcomes this major limitation and can be more effective for improving pregnancy outcomes.

Other tests, such as that prognostic test by Sera Prognostics (Utah, USA) that measure two serum proteins at 17-22 weeks gestation to predict preterm delivery at <37 weeks have low positive predictive values less than 25% and worse negative predictive values. These are not helpful in symptomatic women when knowledge of delivery within 7 days is necessary.

None of the available tests are able to assess the potential of fetal inflammation

The LMA has potential for assessing delivery within 7 days 1) at term in asymptomatic women, 2) at preterm in asymptomatic women, 3) in women at term or preterm with ruptured membranes and 4) at preterm in symptomatic women.

### Specific Details of how the LMA May be Used

The LMA-rytvela approach represents a paradigm shift over other tests for the precise timing of delivery at term and preterm by targeting upstream inflammatory processes leading to PTB using a natural process attendant to every delivery at term or preterm—leukocyte migration. It has the added bonus of predicting FI and it has a highly efficacious intervention—rytvela. The LMA can be applied to five different clinical groups 1) Women symptomatic for PTB. Since 85% of these resolve and deliver at term, the question becomes who to admit to hospital. With a 96% PPV, 60% NPV, 93% Specificity and 75% Sensitivity, the LMA is potentially among the best tests 81 to discriminate who will deliver within 7 days and should be treated with rytvela vs. those who should be sent home. 2) Women asymptomatic for PTB. High-risk women may be tested by the LMA during prenatal visits from 22 wk and treated with rytvela when indicated. 3) Women with ruptured membranes, as they do not all deliver immediately, and rytvela may reduce their risk for FI. 4) Women with chronic infections (chorioamnionitis, HIV, pyelonephritis, Influenza) whose diagnosis could be confirmed by the LMA followed by treatment with rytvela to protect the fetus. 5) Timing of delivery at term. LMA can help schedule a caesarean section or an induction of labour (e.g. for thrombophilia, hemophilia or post-dates).

### Example 4

### 1. Biology of Enhanced Leukocyte Chemotaxis (ELC)

### A. Identification of Key Receptors for Term ELC

We demonstrated that relative mRNA abundance for eight chemokine receptors correlate with the chemotaxis of term leukocytes to term chemoattractant (FIG. 3).

To evaluate whether these receptors are important for the response of term leukocytes to the term chemoattractant, we will compare the response of the leukocytes before and after receptor inhibition. We expect that inhibition of a key receptor will result in fewer term leukocytes migrating in response to the term chemoattractant. Of these eight candidates, the two most interesting ones are CXCR2 and CCR1.

CXCR2 is a major neutrophil chemokine receptor that binds to CXCL1-3 and CXCL5-8. The impairment of neutrophil recruitment is associated with several inflammatory disorders including sepsis, trauma, and obesity. At term parturition in humans and animal models of pregnancy, neutrophils comprise a significant proportion of the leukocytes that infiltrate the gestational tissues. Moreover, Inhibition of CXCR1 and CXCR2 is able to delay preterm delivery and reduce neonatal mortality in a mouse model of chorioamnionitis [1]. As demonstrated in the figure above, the correlation between the mRNA abundance for CXCR2 and the sensitivity of the leukocytes is very significant (p=0.0003).

CCR1 is a broadly expressed chemokine receptor that binds MIP-1α, RANTES, MCP-2/3, and MPIF-1 and is found on monocytes, neutrophils, T-cells and dendritic cells. This gene plays a critical role in the recruitment of effector immune cells to the site of inflammation. CCR1 also induces upregulation of integrins to stimulate firm adhesion of inflammatory cells to the endothelium. In the context of parturition, whole blood mRNA for CCR1 was significantly higher in preterm patients who delivered within two days than those that did not [2]. The correlation between the mRNA abundance for CCR1 and the sensitivity of the leukocytes is very significant (p=0.0009).

We will use SB 225002 (CXCR2 antagonist) and J 113863 (CCR1 antagonist), which are commercially available from RND Systems for around USD 200 each. A time course and kinetics experiment will be performed for each antagonist to optimize incubation length and concentration. TL leukocytes (n=6) will be inhibited with each antagonist using these optimized parameters, and the resulting number of leukocytes that undergo chemotaxis to TL chemoattractant in our LMA will be compared to control (i.e. non-treated leukocytes).

### B. Understanding Term and Preterm ELC

Our data strongly suggests that term and preterm ELC are different at the protein level. Relative mRNA abundance for chemokine receptors is very low at PNL and PL compared to at TNL and/or TL (see below). This suggests that the chemokines that the preterm leukocytes are responding to are different, potentially explaining why leukocyte migration to TL chemoattractant at preterm is different than at term (see right) and why there is no correlation between leukocyte chemotaxis and the mRNA abundance for any of the tested proteins.

Rather, our data suggest that a key protein involved in leukocyte invasion at PL is CX3CR1 (see left). CX3CR1 is a key monocyte recruitment protein that was suggested to play an important role in preterm birth. Macrophage depletion and CX3CR1 knockout were able to mitigate the onset of LPS-induced preterm birth in mice and CX3CR1 antibodies delayed preterm birth in mice. Our lab previously demonstrated that human fetal membranes cocultured with human myometrium produced significantly higher quantities of CX3CL1 following stimulation with IL1β, a central inflammatory mediator of preterm birth [3]. Furthermore, LPS injection at GD15.5 In mice increases mRNA abundance for CX3CL1 in placenta and amnion within 6 h prior to the onset of preterm birth [4].

We will first obtain PL fetal membranes (n=6) from the biobank at CQMU and extract gestational chemoattractant from them. Whole blood will be collected from PL (n=6) and PNL (n=6) patients and their total leukocytes will be isolated. Leukocyte migration to pooled PL and TL fetal membrane extracts will be compared by LMA. CX3CL1 and CXCL8 concentrations will be measured in PL and TL fetal membrane extracts by ELISA. We expect that PNL leukocyte migration to PL extracts will be lower than PL leukocyte migration to these extracts, and that CX3CL1 but not CXCL8 will be higher in the PL extracts than in the TL extracts.

We will use JMS 17-2 hydrochloride (CX3CR1 antagonist), which is commercially available from RND Systems for around USD 200. A time course and kinetics experiment will be performed to optimize incubation length and concentration. PL. PNL and TL leukocytes (n=6 each) will be inhibited with JMS 17-2 hydrochloride using these optimized parameters, and the resulting number of leukocytes that undergo chemotaxis to PL chemoattractant in our LMA will be compared to control (i.e. non-treated leukocytes).

### 2. The Chemokine Scavenger Hypothesis

Historically, the movement of leukocytes has been understood to be directed largely by the chemokine family. Of the almost 50 mammalian chemokines that have been identified, some of them are homeostatic and regulate basal leukocyte trafficking, while others are inflammatory and direct the migration of leukocytes to inflamed or damaged sites. These chemokines bind to chemokine receptors (CC, CXC, XC, CX3C) that are also homeostatic or inflammatory.

In addition to these classical chemokine receptors, there exists a subfamily of atypical chemokine receptors (ACKR) characterized by promiscuous ligand binding and an apparent inability to signal after ligand-receptor binding. Known ACKRs include Duffy antigen receptor for chemokines (ACKR1), D6/CCBP2 (ACKR2), CXCR7/RDC1 (ACKR3), CCX-CKR/CCRL1/CCR11 (ACKR4), and CCRL2 (ACKR5?). These receptors share a high degree of homology with chemokine receptors and lack structural determinants for Gαi signaling, making them unable to activate canonical G protein-coupled pathways such as cell migration. ACKRs bind chemokines and can act as scavenger receptors, transcytosis receptors for chemokines, and regulators of chemokine gradient formation.

In relation to preterm and term labour, ACKR2 is of particular interest because it is a highly promiscuous receptor capable of biding to the majority of inflammatory CC-chemokines and of internalizing and scavenging its ligands by a β-arrestin-dependent signaling pathway. By doing so, ACKR2 can remove chemokines from inflamed tissues and prevent inappropriate accumulation of inflammatory leukocytes (see below).

Previously mRNA coding for ACKR2 has been detected in peripheral leukocytes, and ACKR2 is known to be highly expressed by trophoblasts in the placenta. I hypothesized that ACKR2 is downregulated during the pro-labour period, impacting leukocyte recruitment in two ways: i) it becomes unable to fulfill an autoregulatory role in leukocytes to scavenge chemokines before they can contact chemokine receptors, and ii) it becomes unable to scavenge chemokines away near or at the placenta. Ultimately, this downregulation of ACKR2 would facilitate leukocyte invasion.

We recently demonstrated preliminary evidence that mRNA abundance for ACKR2 trends to be lower in laboring pregnant women than in non-laboring controls, whether at term (TNL: n=8, TL: n=5) or preterm (PNL: n=9, PL: n=5) (see left). This supports the first part of our hypothesis.

We will first test the second part of this hypothesis by measuring the relative mRNA abundance in the human fetal membranes (Lulu agreed to provide me with these samples). Afterwards, we will raise CD1 mice and measure the expression of ACKR2 in peripheral leukocytes and gestational tissues (uterus, fetal membrane) using RT-qPCR and western blot. We will also consider generating a knockout mouse for ACKR2 using CRISPR-Cas9.

### 3. References

[1], Younis S, et al. (2014) Inhibtion of CXCR 1 and 2 delays preterm delivery and reduces neonatal mortality in a mouse model of chorloamnlonitis. Eur J Immunol 447-457.
[2] Heng Y J, Pennell C E, Chua H N, at al. (2014) Whole blood gene expression profile associated with spontaneous preterm birth in women with threatened preterm labor. PLoS One 9(5): e96901.
[3] Leimert K B, Messer A, Gray T, et al. (2018) Maternal and fetal intrauterine tissue crosstalk promotes proinfammatory amplification and uterine transition. Biol Reprod 100(3): 783-97
[4] Mizoguchi M, Ishida Y, Nosaka M, et al. (2018). Prevention of lipopolysaccharide-induced preterm labor by the lack of CX3CL1-CX3CR1 interaction in mice. PLoS One 13(11): e0207085.

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and al such modification as would be obvious to one skilled in the art are intended to be included within the scope of the following claims.

1. A method of determining the likelihood of a pregnant subject undergoing delivery within about 7 days, comprising:
obtaining peripheral leukocytes from said pregnant subject; and
determining a chemotactic activity of said leukocytes, wherein an increased likelihood of delivery within about 7 days is indicated when said chemotactic activity of said leukocytes is at a level characteristic of a full term pregnancy, wherein determining said chemotactic activity comprises measuring migration of said leukocytes towards a chemoattractant, wherein said chemoattractant is a factor present in a sample comprising hFM obtained from a full term pregnant subject.

2. The method of claim 1, wherein the pregnant subject is at least 35 weeks pregnant.

3. The method of claim 1, wherein the pregnant subject is less than 35 weeks pregnant.

4. The method of claim 1, wherein the pregnant subject is at risk of preterm delivery.

5. The method of claim 1, wherein the pregnant subject has ruptured membranes.

6. The method of claim 1, wherein said leukocyte is a granulocyte, T-lymphocyte, monocyte, macrophage, NK cell, or B-lymphocyte.

7. The method of claim 1, wherein said leukocyte is a neutrophil.

8. The method of claim 1, said sample further comprising IL1β.

9. The method of claim 1, wherein said subject is a human.

10. A method of treating a pregnant subject to minimize complications of a preterm delivery, comprising:
obtaining peripheral leukocytes from said pregnant subject; and
determining a chemotactic activity of said leukocytes, wherein an increased likelihood of delivery within about 7 days is indicated when said chemotactic activity of said leukocytes is at a level characteristic of a full term pregnancy, wherein determining said chemotactic activity comprises measuring migration of said leukocytes towards a chemoattractant, wherein said chemoattractant is a factor present in a sample comprising hFM obtained from a full term pregnant subject; and
administering an IL-1 receptor (IL-1 R) antagonist to said pregnant when there is a likelihood of preterm delivery.

11. The method of claim 10, wherein said IL-1 R antagonist is rytvela.

12. The method of claim 10, wherein there is a likelihood of delivery before about 35 weeks of gestation.

13. The method of claim 10, wherein the pregnant subject is less than 35 weeks pregnant.

14. The method of claim 10, wherein the pregnant subject has ruptured membranes.

15. The method of claim 10, wherein said leukocyte is a granulocyte, T-lymphocyte, monocyte, macrophage, NK cell, or B-lymphocyte.

16. The method of claim 10, wherein said leukocyte is a neutrophil.

17. The method of claim 10, said sample further comprising Il_1β.

18. The method of claim 10, wherein said subject is a human.

19. A method of determining the likelihood of a pregnant subject undergoing delivery within about 7 days, comprising:
obtaining peripheral leukocytes from said pregnant subject; and
obtaining a sample from a pregnant subject; contacting the sample with a reagent to a biomarker, to form a complex between the agent and the biomarker present in the sample;
measuring the complex formed to determine the amount or concentration of said biomarker in the sample; wherein an increased likelihood of delivery within about 7 days is indicated when said chemotactic activity of said leukocytes is at a level characteristic of a full term pregnancy, wherein determining said chemotactic activity comprises measuring migration of said leukocytes towards a chemoattractant, wherein said chemoattractant is a factor present in a sample comprising hFM obtained from a full term pregnant subject.

20. The method of claim 19, wherein the biomarker is CXCR2, CXCR3, CXCR5, CCR1, CCR3, CCR5, CX3CR1 and/or CCR7.

21. The method of claim 19, wherein the biomarker is PI3KCB, PI3KCD, Rac1, Vav1, Arp2, and/or Arp3.

22. The method of claim 19, wherein the sample is a serum sample.

23. The method of claim 19, wherein the pregnant subject is at least 35 weeks pregnant.

24. The method of claim 19, wherein the pregnant subject is less than 35 weeks pregnant.

25. The method of claim 19, wherein the pregnant subject is at risk of preterm delivery.

26. The method of claim 19, wherein the pregnant subject has ruptured membranes.

27. The method of claim 19, wherein said subject is a human.

28. A method of determining the likelihood of a pregnant subject undergoing delivery within about 7 days, comprising:
obtaining peripheral leukocytes from said pregnant subject; and
determining a F-actin polarization of said leukocytes, wherein an increased likelihood of delivery within about 7 days is indicated when said F-actin polarization of said leukocytes is at a level characteristic of a full term pregnancy.

29. The method of claim 28, wherein the pregnant subject is at least 35 weeks pregnant.

30. The method of claim 28, wherein the pregnant subject is less than 35 weeks pregnant.

31. The method of claim 28, wherein the pregnant subject is a risk of preterm delivery.

32. The method of claim 28, wherein the pregnant subject has ruptured membranes.

33. The method of claim 28, wherein said leukocyte is a granulocyte, T-lymphocyte, monocyte, NK cell, or B-lymphocyte.

34. The method of claim 28, wherein said leukocyte is a neutrophil.

35. The method of claim 28, wherein said subject is a human.
